SEQUENCE LISTING (73 sequences)

SEQ ID NO: 1 - DNA, 1353 bp, Solanum lycopersicum
  source 1..1353: mol_type = other DNA
atgtctaattttcatgttgctgttatagcatttccttttgcaacacatgctgggcttttatatggacttgttcaaaggctagcaagtacattaccaaatgtgacattcacattttttagcacaaaaaattcgaattcttcgattttcaattcacaacattgttatagcaatacaaatgttaagccctacgacgtctctgatggcgtcccaaagggtcacgtgctcggaggtggtctcgaggagattataggattgttcttcaaatccgcgaagcaaaatattgataatgctatgatttctgctgagaatgactcagggaaaaaaattacatgtgttattgctgatgcatttatgtggtttactagtgatatggctcaacagtgtaatgttcattggattcctgtatggacttctgcagctggatcactatctgttcatgttgatactgatcttattagagaaaatgttgggatacaaggaattgatggacgtgaagatgaagttttgaattttattcctggaatttctgaagtaagacttggtgatttgcctggtggagttgtttctggtgacctaaaatcgccattttcgatgatgcttcataaaatgggaaaaaccataggaaaaggaaaagccatatcaataaattcatttgaagagctagatcctcaaatggttcaatatctcaagtctaaattcaacaacattcttaatgttggtccttttaacttaacttcaccaccacctttagcaaatgttgtagacaaatatgattgtattgagtggttaaatcatcaagagccaaactctgtggcatatattggatttggtactgttgcaacaccaccacctaatgagttagaagcaatggctcaagcacttgaagacaaaaaaattccatttctttggtcaattaaagagaattttatgtcacattttccaaaaggattcttggaaaagacaagtgagtatggtaaagttgtaccatgggtaccacaagtacaagttcttgaacatagtgccgttggagtttttataaatcactctggatggaattcagtactcgagagtatagcgtctggggtacctataatttgtaggccttttttcggtgatcatcatttgaatgcttggatggttgaaaaagtttggaaaattggagtgaaaattgaaggtggggttttcactaaaagtggtacaattgatgcacttgaatttgttttgtcaaaaaataaagaggaattgaaggaacaaattgggatgtataaagaacttgctttaaaggctgttggaacaagtgggagttcaactcaaaatttcaacaagttggttgagattatcacttcttgttag

SEQ ID NO: 2 - DNA, 2840 bp, Solanum lycopersicum
  source 1..2840: mol_type = other DNA
tttactctctccatattaatttttattctattatctccataaactcctctaagaaattaaaaaaaaaaacacaaaaaaatgtctaattttcatgttgctgttatagcatttccttttgcaacacatgctgggcttttatatggacttgttcaaaggctagcaagtacattaccaaatgtgacattcacattttttagcacaaaaaattcgaattcttcgattttcaattcacaacattgttatagcaatacaaatgttaagccctacgacgtctctgatggcgtcccaaagggtcacgtgctcggaggtggtctcgaggagattataggattgttcttcaaatccgcgaagcaaaatattgataatgctatgatttctgctgagaatgactcagggaaaaaaattacatgtgttattgctgatgcatttatgtggtttactagtgatatggctcaacagtgtaatgttcattggattcctgtatggacttctgcagctggatcactatctgttcatgttgatactgatcttattagagaaaatgttgggatacaaggtatacatatatattattcgtcgaaatgacagaatccataaagattattcaatagttgaacatcattgtgtttttctatccattatgaatgaaatgggaaaaatgacagatatacccctgaaatatcgtaaatttatatgcatatatatatatatatatatatatatatatatatatatattctctgttatacttttggaacattgatgcgtctgttatacaaaagttagagcatatatgcccttcactctgacgaaagactgattatggatacgtggcgtaatcttatccgtcgatccgatatttaataaaggtatagtacatagatatgccctttaactttgcctcatttcacatttataccctccaaatttgggtgttcacaagtagacacttaaacttgtatgaacaaatagacacaaacgtcctgtatgacatcctacatgaaaatttgtgtcatacgtgtacaagtttgagtgcctatttgtgcacactcaaagttggagggcacaaatgtaaaatgaggtaaagttaaagggtatatttatgtattatacctttaataaatgtcgggtcggtagataagattatgacacatgtatgtccgttagtttgaagggtatatatactttatttttttatggtaagagcaccaatgtctcaaaagtatgacataaggtatttgcatcaaagacaccaacaatgtcccaaaagtataacagggtatctacataccatttttcaatagtttgagggtacatttgtcctttattcccctgagtatgaaattgactgatattatgacgataataggtaggatagatatcactataggaaaaagttgatttcaaaatgataatcaatacaatataaataaagatctagtatatgaagatatgttgatatttctgaagtacgatctaacttttcttttgatttattacatgttgtattagcgcgatacgataaaaatcattcatttattttttgttatttatagagaactttgtcatgttcatgatgactaaacattggatgaatgattaagactatagtatatgaagatatgttgatgtttctgaagtacgatctaatttttcttttgatttattacatattgtattagcgcgatacaataaaaatcattcatttttttttgtttgataggaattgatggacgtgaagatgaagttttgaattttattcctggaatttctgaagtaagacttggtgatttgcctggtggagttgtttctggtgacctaaaatcgccattttcgatgatgcttcataaaatgggaaaaaccataggaaaaggaaaagccatatcaataaattcatttgaagagctagatcctcaaatggttcaatatctcaagtctaaattcaacaacattcttaatgttggtccttttaacttaacttcaccaccacctttagcaaatgttgtagacaaatatgattgtattgagtggttaaatcatcaagagccaaactctgtggcatatattggatttggtactgttgcaacaccaccacctaatgagttagaagcaatggctcaagcacttgaagacaaaaaaattccatttctttggtcaattaaagagaattttatgtcacattttccaaaaggattcttggaaaagacaagtgagtatggtaaagttgtaccatgggtaccacaagtacaagttcttgaacatagtgccgttggagtttttataaatcactctggatggaattcagtactcgagagtatagcgtctggggtacctataatttgtaggccttttttcggtgatcatcatttgaatgcttggatggttgaaaaagtttggaaaattggagtgaaaattgaaggtggggttttcactaaaagtggtacaattgatgcacttgaatttgttttgtcaaaaaataaagaggaattgaaggaacaaattgggatgtataaagaacttgctttaaaggctgttggaacaagtgggagttcaactcaaaatttcaacaagttggttgagattatcacttcttgttagtgaaactataatatattcttgaaaaggggttttaatgattaattattaattccacttttttttttttttaaaaaaatatatatatatatatgtatcaattgttatcatgaattgatcatcaatatttgtccatgtaacattgtttttgggaaaacatttgtaatgaaattgaaactcatgactttctagtcacacaacaaactcttaccattgcatcaatataatatactctctctcgttaa

SEQ ID NO: 3 - protein, 450 aa, Solanum lycopersicum
MSNFHVAVIAFPFATHAGLLYGLVQRLASTLPNVTFTFFSTKNSNSSIFNSQHCYSNTNVKPYDVSDGVPKGHVLGGGLEEIIGLFFKSAKQNIDNAMISAENDSGKKITCVIADAFMWFTSDMAQQCNVHWIPVWTSAAGSLSVHVDTDLIRENVGIQGIDGREDEVLNFIPGISEVRLGDLPGGVVSGDLKSPFSMMLHKMGKTIGKGKAISINSFEELDPQMVQYLKSKFNNILNVGPFNLTSPPPLANVVDKYDCIEWLNHQEPNSVAYIGFGTVATPPPNELEAMAQALEDKKIPFLWSIKENFMSHFPKGFLEKTSEYGKVVPWVPQVQVLEHSAVGVFINHSGWNSVLESIASGVPIICRPFFGDHHLNAWMVEKVWKIGVKIEGGVFTKSGTIDALEFVLSKNKEELKEQIGMYKELALKAVGTSGSSTQNFNKLVEIITSC

SEQ ID NO: 4 - DNA, 1359 bp, Nicotiana tabacum
  source 1..1359: mol_type = other DNA
atgtccaattatcatgttgctgttctagcattcccttttgcaacacatgctggccttttacatggacttgtccaaaggctagcaaatacattacctaatgtaacattcacatttttcaacacctccaaatcaaattctttaattttctccaatcaaaccgacccacattgtactaatcttaagcccttcgatatctccgatggcgtacccgagggatacgcgatcggagaaggaggaatcgaggagcttatagggttattttttaaatccgcgaaaaataattttcagaatgctatatcagctgctgaggatgaaacgggaaaaaaaattacgtgcgttttcgcggatgcgtttatatggttttccggtgagattgcggaggaattaggcgttgattggattcctgtttggacttctgcggcaggatcactgtctgttcatatttacactgattttattagagaaaatgttggggttcaaggaattgctggacgtgaagatgaaatgttaacattcattcctggattttctgaattaaggcttggtgatttgcctagtggagttatatttggagatttaaaatcaccattttctaaaatgcttcacaaaatgggcaaaaccataacaaaagccaatgccttaccaataaattcctttgaagaattagaccctccaattgttgaaaatctcaagtccaaattcaataattttcttaatgttggtccttttaacttaacctccccaccaccctcagcaaatattatagacaaaaatggttgtattgaatggctagataatcaagaaccaaattctgtggcttatattggatttggcacagttgcaacaccaccacctaatgagttaaaatctatggctgaagcacttgaagaaagtaagacaccttttctttggtcaattaaagatgattttatatcacattttccaaaagaatttttggaaaatacaagtgagtatggtaaaattgtaacatgggccccacaagtacaagttcttgaacatagttcaattggggtttttataaatcatagtggatggaattctgtgttggaaagtatagcttctggtgtgccaataatttgtaggccattttttggagaccatcatttgaattcttggatggttgaaaaagtgtggaaaattggagtgaaaattgaaggtggggtttttactaaaagtggtacaatggttgcacttgacttggttttatcaaaaaatagagagaaattgaagcaacaaattggtatatataaagatcttgctctcaaggctgttggaccaagtgggagttcaactgaaaatttcaagaaattggttgagattatcacctctagcaattga

SEQ ID NO: 5 - protein, 452 aa, Nicotiana tabacum
MSNYHVAVLAFPFATHAGLLHGLVQRLANTLPNVTFTFFNTSKSNSLIFSNQTDPHCTNLKPFDISDGVPEGYAIGEGGIEELIGLFFKSAKNNFQNAISAAEDETGKKITCVFADAFIWFSGEIAEELGVDWIPVWTSAAGSLSVHIYTDFIRENVGVQGIAGREDEMLTFIPGFSELRLGDLPSGVIFGDLKSPFSKMLHKMGKTITKANALPINSFEELDPPIVENLKSKFNNFLNVGPFNLTSPPPSANIIDKNGCIEWLDNQEPNSVAYIGFGTVATPPPNELKSMAEALEESKTPFLWSIKDDFISHFPKEFLENTSEYGKIVTWAPQVQVLEHSSIGVFINHSGWNSVLESIASGVPIICRPFFGDHHLNSWMVEKVWKIGVKIEGGVFTKSGTMVALDLVLSKNREKLKQQIGIYKDLALKAVGPSGSSTENFKKLVEIITSSN

SEQ ID NO: 6 - DNA, 1359 bp, Capsicum annuum
  source 1..1359: mol_type = other DNA
atgtctaataatttacatgttgctgttcttgcatttccatttgcaacacatgctggtcttttactaggacttatacaaaggctatcaaattcattaccaaatgttacatttacatttttcaatactaaaagatcaaattcttcaatatttacacaagataataatacaaatattaagccatttgatatccctgatgggaaacctgagagtcatgtacatggaggtggagatgaaggtggactcgaagatattataggtttattcttcaaatccgcgaaaaataatgtgacaaatgccatcgcgactgctgagaatgactcagggaagaagattacgtgtgtcatagctgacgcgtttatgtggttttcgagtgagatcaccgaggaactaggaattagttggattccagtatggacttccgcggcaggatcactctctgtgcatgttcataccgatcttattagagaaaacgttgggacccaaggaattgatggacgcgaagatgaaattttgaaatttattccgggttttttcgaagtaagacttggcgatttacctggaggagttgtcgtaggagatctgaaaacgtcattttctatgatgcttcacaaaatgggccaaacaataggtaaaggaacagccataccaataaattcatttgaagaattggaccctccaattgtccaaaatctgaaatccaaattcaaaaattttctcaatattggaccatttaatttaacttcaccaacaccattagcaaatgtggttgacaaagatggttgtattgaatggttagacaaacaaaaaactaattctgtggcatatattggatttggcactgtagcaacaccaccacctaatgagttaaaagcaatggctgaagcacttgaagagaaaaaaataccatttctatggtcaattaaagagaaatttattgcaaattttccaaatggattcttggaaaaaacaaaaatgtttggtaaagttgtgtcatgggcaccacaagtacaagttcttgaacataaagcgatcggcgtttttataaatcactcaggatggaattcggttctggagagcatagcttctggcgtgcctataatatgtaggccgtttttcggagatcatcatttgaattcttggatggttgagaaagtttggaaaattggtgtgaaaattgaaggtggggtttttactaaaagtggtacaattgttgcacttgatttggttttgtctaaaaataaagaagaatttaagaaacaaattggaatgtacaaagaattagttcacaatgctgttggaccaaatggaagttcaacgcaaaatttcaagaaattggttgagattatcacctcttgttag

SEQ ID NO: 7 - protein, 452 aa, Capsicum annuum
MSNNLHVAVLAFPFATHAGLLLGLIQRLSNSLPNVTFTFFNTKRSNSSIFTQDNNTNIKPFDIPDGKPESHVHGGGDEGGLEDIIGLFFKSAKNNVTNAIATAENDSGKKITCVIADAFMWFSSEITEELGISWIPVWTSAAGSLSVHVHTDLIRENVGTQGIDGREDEILKFIPGFFEVRLGDLPGGVVVGDLKTSFSMMLHKMGQTIGKGTAIPINSFEELDPPIVQNLKSKFKNFLNIGPFNLTSPTPLANVVDKDGCIEWLDKQKTNSVAYIGFGTVATPPPNELKAMAEALEEKKIPFLWSIKEKFIANFPNGFLEKTKMFGKVVSWAPQVQVLEHKAIGVFINHSGWNSVLESIASGVPIICRPFFGDHHLNSWMVEKVWKIGVKIEGGVFTKSGTIVALDLVLSKNKEEFKKQIGMYKELVHNAVGPNGSSTQNFKKLVEIITSC

SEQ ID NO: 8 - DNA, 1356 bp, Solanum tuberosum
  source 1..1356: mol_type = other DNA
atgtctaattatcatgttgctgttatagcatttccttttgcaacacatgctggccttttatatggacttgttcaaaggctagcaaatacattaccaaatgtgacattcacattttttagcacaaaaatttcgaattcttcaatttttaattcacaaaattataattatagaaatacaaatcttaagccctatgacgtctctgatggcgtcccagagggtcacgtgctcggaggtggtctcgaagagattataggattattcttcaaatccgcgaagaaaaatattcataatgctataatttctgctgagaatgactcagggaaaaaaattacatgtgttattgcggacgcgtttatgtggttttcgagtaatatggctgaagagtttaacgttagttggattcctgtatggacttccgcagctggatcactctctgttcatgttgataccgatgttataagacaaaatgttgggatccaaggaattgatggacgcgaagatgaaattttgaaatttattcctggaatttctgaagtaagacttggtaatttgcctggtggaattgtttctggagacctaaaatcgccattttcgatgatgcttcataaaatgggaaaaaccataggaaaaggaaaagccataccaataaattcatttgaagaattagaccctcaaatggttcaatatctcaagtctaaattcaacaatattcttaatgttggtccttttaatttaacttcaccaccacctctagcaaatgttgtagacaaatatgattgtattgagtggttaaatagtcaagaaccaaattctgtggcatatattggatttggtactgttgcaacaccaccacctaatgagttaaaagctatggctcaagcacttgaagaaaaaaaaattccatttatttggtcaattaaagagaattttatgtcacattttccacaagggttcttggaaaacacaagtgagtatggtaaagttgtaccatgggtaccacaagtacaagttcttgaacatagtgcgattggagtttttataaatcactctggatggaattcggttctggagagcatagcttctggggtgcctataatttgtaggcctttttttggagatcatcatttgaatgcttggatggttgagaaagtttggaaaattggtgtgaaaattgaaggtggggtttttacaaaaagtggtacaattgatgcacttgacttggttttgtcaaaaaatagagtggaattgaaggaacaaattggaatgtacaaagaacttgctttaaaggctgttggaacatgtgggagttcaactcaaaatttcaacaagttggttgagattatcacttcttgttaa

SEQ ID NO: 9 - protein, 451 aa, Solanum tuberosum
MSNYHVAVIAFPFATHAGLLYGLVQRLANTLPNVTFTFFSTKISNSSIFNSQNYNYRNTNLKPYDVSDGVPEGHVLGGGLEEIIGLFFKSAKKNIHNAIISAENDSGKKITCVIADAFMWFSSNMAEEFNVSWIPVWTSAAGSLSVHVDTDVIRQNVGIQGIDGREDEILKFIPGISEVRLGNLPGGIVSGDLKSPFSMMLHKMGKTIGKGKAIPINSFEELDPQMVQYLKSKFNNILNVGPFNLTSPPPLANVVDKYDCIEWLNSQEPNSVAYIGFGTVATPPPNELKAMAQALEEKKIPFIWSIKENFMSHFPQGFLENTSEYGKVVPWVPQVQVLEHSAIGVFINHSGWNSVLESIASGVPIICRPFFGDHHLNAWMVEKVWKIGVKIEGGVFTKSGTIDALDLVLSKNRVELKEQIGMYKELALKAVGTCGSSTQNFNKLVEIITSC

SEQ ID NO: 10 - DNA, 20 bp, synthetic construct
  source 1..20: mol_type = other DNA
ccaaagggtcacgtgctcgg

SEQ ID NO: 11 - DNA, 20 bp, synthetic construct
  source 1..20: mol_type = other DNA
gtacccgagggatacgcgat

SEQ ID NO: 12 - DNA, 20 bp, synthetic construct
  source 1..20: mol_type = other DNA
ttctaaaatgcttcacaaaa

SEQ ID NO: 13 - DNA, 20 bp, synthetic construct
  source 1..20: mol_type = other DNA
ggtggactcgaagatattat

SEQ ID NO: 14 - DNA, 20 bp, synthetic construct
  source 1..20: mol_type = other DNA
tttccatttgcaacacatgc

SEQ ID NO: 15 - DNA, 20 bp, synthetic construct
  source 1..20: mol_type = other DNA
ggtggtctcgaagagattat

SEQ ID NO: 16 - DNA, 20 bp, synthetic construct
  source 1..20: mol_type = other DNA
atgggaaaaaccataggaaa

SEQ ID NO: 17 - DNA, 20 bp, synthetic construct
  source 1..20: mol_type = other DNA
atttgggatggcgtcaaaca

SEQ ID NO: 18 - DNA, 21 bp, synthetic construct
  source 1..21: mol_type = other DNA
agacctcaaatcaaagctcca

SEQ ID NO: 19 - DNA, 21 bp, synthetic construct
  source 1..21: mol_type = other DNA
agtcttgactccgttgtctct

SEQ ID NO: 20 - DNA, 21 bp, synthetic construct
  source 1..21: mol_type = other DNA
agcatttccttttgcaacaca

SEQ ID NO: 21 - DNA, 20 bp, synthetic construct
  source 1..20: mol_type = other DNA
agtgatccagctgcagaagt

SEQ ID NO: 22 - DNA, 20 bp, synthetic construct
  source 1..20: mol_type = other DNA
aaacaaggcaatgacacgcc

SEQ ID NO: 23 - DNA, 20 bp, synthetic construct
  source 1..20: mol_type = other DNA
ccctgtttcctcctctgctt

SEQ ID NO: 24 - DNA, 19 bp, synthetic construct
  source 1..19: mol_type = other DNA
gacttctgcagctggatca

SEQ ID NO: 25 - DNA, 20 bp, synthetic construct
  source 1..20: mol_type = other DNA
aactccaccaggcaaatcac

SEQ ID NO: 26 - DNA, 20 bp, synthetic construct
  source 1..20: mol_type = other DNA
ttgctgaccgtatgagcaag

SEQ ID NO: 27 - DNA, 20 bp, synthetic construct
  source 1..20: mol_type = other DNA
ggacaatggatggaccagac

SEQ ID NO: 28 - DNA, 22 bp, synthetic construct
  source 1..22: mol_type = other DNA
cgatgtgtgatctcctatggtc

SEQ ID NO: 29 - DNA, 22 bp, synthetic construct
  source 1..22: mol_type = other DNA
aagctgatgggctctagaaatc

SEQ ID NO: 30 - protein, 22 aa, Camellia sinensis
IGVLAFPFGTHAAPLLTLVRRL

SEQ ID NO: 31 - protein, 17 aa, Camellia sinensis
PWVPYWTAGSCSLSIHF

SEQ ID NO: 32 - protein, 16 aa, Camellia sinensis
VPMICRPFFGDQTLNS

SEQ ID NO: 33 - protein, 22 aa, Petunia sp.
IALLAFPFGSHAAPLLTLVQKL

SEQ ID NO: 34 - protein, 17 aa, Petunia sp.
PWIAFWTAASGSLSVHL

SEQ ID NO: 35 - protein, 16 aa, Petunia sp.
VPMICRPFFGDQKLNS

SEQ ID NO: 36 - protein, 22 aa, Nicotiana tabacum
IALLAFPFGSHATPLLTLVQKL

SEQ ID NO: 37 - protein, 17 aa, Nicotiana tabacum
PWIAFWTAGSGSLSVHL

SEQ ID NO: 38 - protein, 16 aa, Nicotiana tabacum
VPMICRPFFGDQKLNS

SEQ ID NO: 39 - protein, 22 aa, Capsicum annuum
IAVLAFPFGSHAVPLLTLVQKI

SEQ ID NO: 40 - protein, 17 aa, Capsicum annuum
PWIAFWTAGSCSLSVHL

SEQ ID NO: 41 - protein, 16 aa, Capsicum annuum
VPMICRPSFGDQKLNS

SEQ ID NO: 42 - protein, 22 aa, Solanum tuberosum
IALLAFPFGSHAAPLLTLVQKI

SEQ ID NO: 43 - protein, 17 aa, Solanum tuberosum
PWIAFWTAGSCSLSIHL

SEQ ID NO: 44 - protein, 16 aa, Solanum tuberosum
VPMICRPFFGDQKLNS

SEQ ID NO: 45 - protein, 22 aa, Solanum lycopersicum
IALLAFPFGSHAAPLLTLAQKI

SEQ ID NO: 46 - protein, 17 aa, Solanum lycopersicum
PWIAFWTAGSCSLSIHL

SEQ ID NO: 47 - protein, 16 aa, Solanum lycopersicum
VPMICRPFFGDQKLNS

SEQ ID NO: 48 - protein, 22 aa, Camellia sinensis
IAVLAFPFASHAGLTLGLIRRL

SEQ ID NO: 49 - protein, 17 aa, Camellia sinensis
PWVPLWTSGACSLSTHC

SEQ ID NO: 50 - protein, 16 aa, Camellia sinensis
VPMIGRPFFGDHHINT

SEQ ID NO: 51 - protein, 22 aa, Petunia sp.
VAVLAFPFATHAGLLLGLVQRL

SEQ ID NO: 52 - protein, 17 aa, Petunia sp.
GWIPLWTSAAGSLSVHV

SEQ ID NO: 53 - protein, 16 aa, Petunia sp.
VPVICRPFFGDHQLNA

SEQ ID NO: 54 - protein, 22 aa, Nicotiana tabacum
VAVLAFPFATHAGLLHGLVQRL

SEQ ID NO: 55 - protein, 17 aa, Nicotiana tabacum
DWIPVWTSAAGSLSVHI

SEQ ID NO: 56 - protein, 16 aa, Nicotiana tabacum
VPIICRPFFGDHHLNS

SEQ ID NO: 57 - protein, 22 aa, Capsicum annuum
VAVLAFPFATHAGLLLGLIQRL

SEQ ID NO: 58 - protein, 17 aa, Capsicum annuum
SWIPVWTSAAGSLSVHV

SEQ ID NO: 59 - protein, 16 aa, Capsicum annuum
VPIICRPFFGDHHLNS

SEQ ID NO: 60 - protein, 22 aa, Solanum tuberosum
VAVIAFPFATHAGLLYGLVQRL

SEQ ID NO: 61 - protein, 17 aa, Solanum tuberosum
SWIPVWTSAAGSLSVHV

SEQ ID NO: 62 - protein, 16 aa, Solanum tuberosum
VPIICRPFFGDHHLNA

SEQ ID NO: 63 - protein, 22 aa, Solanum lycopersicum
VAVIAFPFATHAGLLYGLVQRL

SEQ ID NO: 64 - protein, 17 aa, Solanum lycopersicum
HWIPVWTSAAGSLSVHV

SEQ ID NO: 65 - protein, 16 aa, Solanum lycopersicum
VPIICRPFFGDHHLNA

SEQ ID NO: 66 - DNA, 28 bp, synthetic construct
  source 1..28: mol_type = other DNA
acatttgggatggcgtcaaacaaggcaa

SEQ ID NO: 67 - DNA, 34 bp, synthetic construct
  source 1..34: mol_type = other DNA
tcccaaagggtcacgtgctcggaggtggtctcga

SEQ ID NO: 68 - DNA, 24 bp, synthetic construct
  source 1..24: mol_type = other DNA
acatttgggatggcgtcaaggcaa

SEQ ID NO: 69 - DNA, 34 bp, synthetic construct; identical to SEQ ID NO: 67
  source 1..34: mol_type = other DNA

SEQ ID NO: 70 - DNA, 19 bp, synthetic construct
  source 1..19: mol_type = other DNA
acatttgggatggcggcaa

SEQ ID NO: 71 - DNA, 32 bp, synthetic construct
  source 1..32: mol_type = other DNA
tcccaaagggtcacgtgctcaggtggtctcga

SEQ ID NO: 72 - DNA, 24 bp, synthetic construct
  source 1..24: mol_type = other DNA
acatttgggatggcgtcaaggcaa

SEQ ID NO: 73 - DNA, 12 bp, synthetic construct
  source 1..12: mol_type = other DNA
tcccaaagtcga

(19) United States
(12) Patent Application Publication
(10) Pub. No.: US 2026/0286380 A1
(43) Pub. Date: Sep. 24, 2026

(54) CONTROL OF MALE FERTILITY IN CROPS

(71) Applicant: University of Florida Research Foundation, Incorporated, Gainesville, FL (US)

(72) Inventors: Jeongim KIM, Gainesville, FL (US); Doosan SHIN, Gainesville, FL (US)

(21) Appl. No.: 19/574,549

(22) Filed: Mar. 23, 2026

Related U.S. Application Data
(60) Provisional application No. 63/776,657, filed on Mar. 24, 2025.

(51) Int. Cl. C12N 15/82; C12N 9/10; C12N 9/22; C12N 15/11

(52) U.S. Cl. CPC C12N 15/8289; C12N 9/1051; C12N 9/224; C12N 9/226; C12N 15/111; C12N 15/8213; C12Y 204/01091

(57) ABSTRACT

Genetically modified plants comprising a modified UGT78D-B gene are described. The genetically modified plants have reduced male fertility. Also described at compositions for making the genetically modified plants as well as methods of making the genetically modified plants.

### CROSS-REFERENCE TO RELATED APPLICATIONS

This application claims the benefit of U.S. Provisional application 63/776,657, filed Mar. 24, 2025, which is incorporated herein by reference.

### GOVERNMENT SUPPORT

This invention was made with government support under IOS-2142898 awarded by the National Science Foundation. The government has certain rights in the invention.

### SEQUENCE LISTING

The Sequence Listing written in file T19696US002_SeqListing.xml is 74,970 bytes in size, was created Mar. 4, 2026, and is hereby incorporated by reference.

### INTRODUCTION

Flavonoids are specialized metabolites widely distributed throughout the plant kingdom. The core structure of flavonoids, a C6-C3-C6 skeleton, comprises two aromatic rings (A and B) and a heterocyclic ring (C). Depending on the structure of the C-ring, flavonoids are further categorized into subclasses, namely flavones, flavonols, flavanones, flavanols, anthocyanidins, and isoflavones.

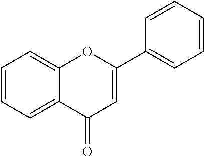 [SMILES: O=c1cc(-c2ccccc2)oc2ccccc12]

Flavonols are a subclass of flavonoids, with hundreds of structures commonly found in plants. This chemical diversity primarily arises from glycosylation, where different sugars are selectively added to various positions on the flavonol aglycones. Flavonol, in particular, is characterized by its 3-hydroxyflavone backbone, which can be further modified through hydroxylation, methylation, acylation, and glycosylation resulting in various flavonol structures.

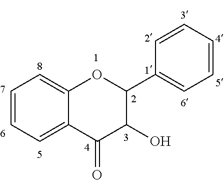 [SMILES: O=C1c2ccccc2OC(c2ccccc2)C1O]

Among these modifications, glycosylation significantly contributes to the structural diversity of flavonols due to the various types, positions, and numbers of sugar units attached to the aglycones. Glycosylation is primarily catalyzed by UDP-sugar-dependent glycosyltransferases (UGTs), which transfer sugar moieties to hydroxyl groups on flavonols. Kaempferol and quercetin differ in the presence or absence of a hydroxyl group at the 3′ position of the B-ring, a modification mediated by flavonoid 3′-hydroxylase (F3′H).

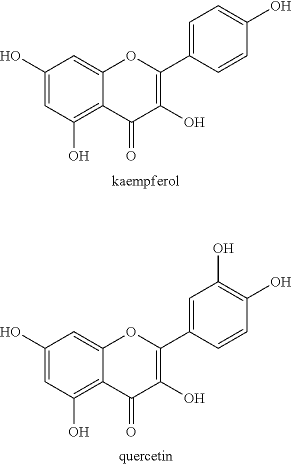 [SMILES: O=c1c(O)c(-c2ccc(O)cc2)oc2cc(O)cc(O)c12 ; O=c1c(O)c(-c2ccc(O)c(O)c2)oc2cc(O)cc(O)c12]

### SUMMARY

Described are compositions and methods for modifying male fertility in a plant. The methods comprising modifying activity of a pollen-specific flavonol 3-O-glycosyltransferase (F3GT) in the plant. In some embodiments, the methods comprise modifying expression of a F3GT gene in the plant. In Solanum lycopersicum, the pollen-specific F3GT gene is encoded by the SIUGT78D-B gene. In other plant species, the pollen-specific F3GT gene (hereafter called the UGT78D-B gene) is an ortholog of SIUGT78D-B. In some embodiments, modifying male fertility in plants comprises modifying pollen tube growth. In some embodiments, modifying pollen tube growth comprises modifying pollen tube length. In some embodiments, the methods of reducing male fertility or reducing pollen tube growth or length in a plant comprise reducing (e.g., knocking down) or knocking out expression of the UGT78D-B gene (e.g., the SIUGT78D-B gene or an ortholog thereof in another plant species) in the plant.

In some embodiments, reducing male fertility results in reduced seed yield. In some embodiments, seed yield is reduced without substantially altering fruit production. In some embodiments, seed yield is reduced without substantially altering fruit size. In some embodiments, seed yield is reduced without substantially reducing the number of fruits produced per plant. In some embodiments, seed yield is reduced without substantially reducing fruit size or the number of fruits produced per plant.

In some embodiments, decreasing expression of UGT78D-B in a plant does not substantially affect morphology of pollen grains produced by the plant.

In some embodiments, modifying activity of UGT78D-B comprises reducing expression of a gene encoding the UGT78D-B. In some embodiments, reducing expression of the UGT78D-B expression comprises reducing mRNA encoding the UGT78D-B mRNA. In some embodiments, reducing expression of the UGT78D-B comprises introducing an RNA interference (RNAi) molecule or an antisense nucleotide targeting the UGT78D-B mRNA into the plant or expressing an RNAi nucleic acid sequence or an antisense nucleic acid sequence targeting the UGT78D-B mRNA in the plant. In some embodiments, reducing the UGT78D-B mRNA comprises modifying an endogenous gene encoding a UGT78D-B mRNA (i.e., an UGT78D-B gene) to create a deletion, insertion, substitution, or frameshift mutation in the endogenous gene encoding the UGT78D-B.

In some embodiments, modifying activity of the UGT78D-B comprises introducing a mutation into an endogenous gene encoding the UGT78D-B (UGT78D-B gene) that results in reduced levels or loss of function of the encoded UGT78D-B.

In some embodiments, modifying activity of the UGT78D-B comprises introducing a CRISPR system into a plant cell, wherein the CRISPR system comprises an RNA-guided DNA endonuclease or a nucleic acid encoding the RNA-guided DNA endonuclease and a guide RNA or a nucleic acid encoding the guide RNA into the plant, wherein the RNA-guided DNA endonuclease and the guide RNA form a complex that targets a UGT78D-B genomic locus. In some embodiments, the CRISPR system is selected from the group consisting of: a CRISPR class 1 system, a CRISPR class 2 system, a CRISPR/Cas system, a CRISPR/Cas9 system, a CRISPR/zCas9 system and a CRISPR/Cas3 system. In some embodiments, the RNA-guided endonuclease comprises a Cas9 endonuclease.

In some embodiments, the guide RNA comprises a CRISPR RNA (crRNA) and a trans-activating CRISPR RNA (tracrRNA) as separate molecules or as a single chimeric guide RNA (sgRNA).

In some embodiments, we describe CRISPR systems that target a UGT78D-B genomic locus (i.e., a UGT78D-B gene; i.e., an SIGT78D-B gene or an ortholog thereof in another plant).

The described CRISPR systems can be introduced into plants using methods available in the art. In some embodiments, introducing a CRISPR system into a plant cell comprises electroporation, microprojectile bombardment, biolistic transformation, microinjection, protoplast transformation, an Agrobacterium tumefaciens vector transformation or an Agrobacterium rhizogenes vector transformation.

In some embodiments, the CRISPR system introduces a mutation into the genomic UGT78D-B locus that results in loss of enzymatic activity of the encoded UGT78D-B or reduces expression of the UGT78D-B gene. In some embodiments, the mutation comprises a deletion, insertion, substitution, or frameshift mutation, or combinations thereof, in the encoded UGT78D-B or its promoter region. In some embodiments, the mutation comprises a deletion of one (1) or more nucleotides or an insertion one (1) or more nucleotides in the UGT78D-B genomic locus that results in decreased expression of the UGT78D-B gene. In some embodiments, the mutation comprises a deletion in one (1) or more nucleotides or an insertion one (1) or more nucleotides in the UGT78D-B genomic locus that results in reduced activity or loss of activity of the encoded UGT78D-B. In some embodiments, the mutation comprises a substitution of one (1) or more amino acids in the encoded UGT78D-B that results in reduced activity or loss of activity of the encoded UGT78D-B.

In some embodiments, a CRISPR system targets introduces a mutation in the UGT78D-B gene that results in premature termination of translation of the UGT78D-B mRNA.

Also described are genetically modified plants made using the described methods, including progeny of such plants. In some embodiments, the plants have modified male fertility. In some embodiments, the plants have reduced male fertility.

Also described are genetically modified plants having reduced male sterility, wherein the genetically modified plants contain a modified UGT78D-B gene.

In some embodiments, genetically modified plants having a modified UGT78D-B gene have modified male fertility relative to a similar plant that does not have a genetic modification of an endogenous UGT78D-B gene.

In some embodiments, genetically modified plants having a disruption (e.g., deletion, insertion, substitution, frameshift, or nonsense mutation) of the UGT78D-B gene have reduced male fertility relative to similar plants that do not have the disruption of an endogenous UGT78D-B gene. In some embodiments, male fertility of the plants is reduced by at least 20%, at least 25%, at least 30%, at least 35%, at least 40%, at least 45%, at least 50%, at least 60%, at least 65%, at least 70%, at least 75%, at least 80%, at least 85%, or at least 90% relative to similar plants that does not have the disruption of an endogenous UGT78D-B gene.

In some embodiments, genetically modified plants having a disruption (e.g., deletion, insertion, substitution, frameshift, or nonsense mutation, or combinations thereof) of the UGT78D-B gene have reduced pollen tube growth relative to similar plants that do not have the disruption of an endogenous UGT78D-B gene. In some embodiments, pollen tube growth in plants having a disruption (e.g., deletion, insertion, substitution, frameshift, or nonsense mutation, or combinations thereof) of the UGT78D-B gene is reduced by at least 20%, at least 25%, at least 30%, at least 35%, at least 40%, at least 45%, at least 50%, at least 60%, at least 65%, at least 70%, at least 75%, at least 80%, at least 85%, or at least 90% relative to similar plants that do not have the disruption of the endogenous UGT78D-B gene. Pollen tube grown is reduced for pollen originating from the ugt78d-b plant (i.e., a plant having a disruption of the UGT78D-B gene). The genotype of the pistil does not significantly influence pollen tube growth if pollen is from a ugt78d-b plant.

In some embodiments, genetically modified plants having a disruption (e.g., deletion, insertion, substitution, frameshift, or nonsense mutation, or combinations thereof) of the UGT78D-B gene have reduced seed production growth relative to similar plants that do not have the disruption of an endogenous UGT78D-B gene. In some embodiments, seed production in plants having a disruption (e.g., deletion, insertion, substitution, frameshift, or nonsense mutation, or combinations thereof) of the UGT78D-B gene is reduced by at least 20%, at least 25%, at least 30%, at least 35%, at least 40%, at least 45%, at least 50%, at least 60%, at least 65%, at least 70%, at least 75%, at least 80%, at least 85%, or at least 90% relative to a seed production in similar plants that do not have the disruption of the endogenous UGT78D-B gene.

The plant can be, but is not limited to, a crop plant. The crop plant can be, but is not limited to, a blackberry plant, a rice plant, a maize plant, a sorghum plant, a rye plant, a barley plant, a wheat plant, an oat plant, a sugarcane plant, a soybean plant, a canola plant, an alfalfa plant, a sunflower plant, a cotton plant, a tobacco plant, a peanut plant, a tomato plant, or a potato plant.

In some embodiments, the UGT78D-B comprises the amino acid sequence of SEQ ID NO: 3, 5, 7, or 9 or an ortholog thereof. In some embodiments, the UGT78D-B gene encodes an mRNA having the nucleic acid sequence of SEQ ID NO:1 or 2, 4, 6, or 8 or an ortholog thereof.

### BRIEF DESCRIPTION OF THE DRAWINGS

FIG. 1A. Quantification of three major flavonol glycosides (K2, K4 and Q4) across different tomato organs detected with HPLC. The data presented are the mean concentrations (pmol mg-1 fresh weight) of flavonol glycosides and their standard deviations (n=4). Compounds that were below the detection limit in certain organs are marked as not detected (n.d.).

FIG. 1B. Simplified biosynthetic pathway of flavonol glycosides in tomato. Flavonol biosynthesis begins with the conversion of phenylalanine into dihydroflavonols, mediated by several enzymatic steps. Dihydroflavonols are then converted to flavonols (kaempferol and quercetin) by flavonol synthase (FLS). Kaempferol (K) and quercetin (Q) undergo glycosylation by flavonol 3-O-glycosyltransferases (F3GT), leading to the production of initial glycosylated intermediates, such as kaempferol 3-O-galactoside (K-gal; K1), kaempferol 3-O-glucoside (K-glu; K3), quercetin 3-O-galactoside (Q-gal; Q1), and quercetin 3-O-glucoside (Q-glu; Q3). These intermediates are further modified by flavonol 3-O-glycoside: glycosyltransferases (F3GGT), generating secondary glycosides, including kaempferol 3-O-glucosyl (1→2) galactoside (K-gal-glu; K2), kaempferol 3-O-glucosyl (1→6) rhamnoside (K-glu-rha; K4), quercetin 3-O-glucosyl (1→2) galactoside (Q-gal-glu; Q2), and quercetin 3-O-glucosyl (1→6) rhamnoside (Q-glu-rha; Q4). Abbreviations used in chemical structures are K, kaempferol; Q, quercetin; gal, galactose; glu, glucose; rha, rhamnose.

FIG. 2A. In vitro enzymatic assays of S/UGT78D-A and S/UGT78D-B. HPLC chromatograms of enzyme reaction mixtures demonstrate the production of K-glu (K3), Q-glu (Q3), K-gal (K1), and Q-gal (Q1) when S/UGT78D-A and S/UGT78D-B were incubated with two flavonol aglycones, kaempferol (K) and quercetin (Q), and two sugar donors, UDP-glucose and UDP-galactose. S/UGT78D-A shows both glucosyltransferase and galactosyltransferase activities at the 3-OH position on kaempferol and quercetin, while S/UGT78D-B exhibits galactosyltransferase activity, but no glucosyltransferase activity. Chromatograms were obtained at a wavelength of 345 nm.

FIG. 2B. HPLC chromatograms showing the enzymatic activity of S/UGT79B-B, which produces K-gal-glu (K2) from K-gal using UDP-glucose as sugar donor. S/UGT79B-A does not show this activity. The chromatograms were obtained at a wavelength of 345 nm.

FIG. 2C. Working model of transcriptional regulation of key enzymes determining the distinct flavonol glycoside profiles between pollen and other organs. In organs outside of pollen, flavonoid 3′-hydroxylase (F3′H) is active, producing both kaempferol and quercetin, which are glucosylated by S/UGT78D-A to form flavonol 3-O-glucosides (F-glu). Further glycosylation by SZUGT79B-A results in the formation of flavonol 3-O-glucosyl (1→6) rhamnoside (F-glu-rha; Q4, K4). In pollen, however, F3′H expression is repressed, and galactosylation becomes predominant due to the activation of S/UGT78D-B and repression of S/UGT78D-A. This switch leads to the production of kaempferol 3-O-galactoside (K-gal). Further modification by S/UGT79B-B results in kaempferol 3-O-glucosyl (1→2) glucoside (K2), which is uniquely abundant in pollen.

FIG. 3A. Identification of key amino acids for sugar specificity in S/UGT78D-B. Sequence alignment of F3GTs showing four amino acid residues conserved in each group, which are highlighted in red and blue for the S/UGT78D-A group and the S/UGT78D-B group, respectively. Amino acids positions 14, 19, 138, and 373 are indicated based on the S/UGT78D-B sequences. (Sequences: CsF3GluT=SEQ ID NOs: 30-32; PhF3GluT=SEQ ID NOs: 33-35; NtF3GluT=SEQ ID NOs: 36-38; CaF3GluT=SEQ ID NOs: 39-41; S/F3GluT=SEQ ID NOs: 42-44; S/UGT78D-A=SEQ ID NOs: 45-47; CsF3GalT=SEQ ID NOs: 48-50; PhF3GalT=SEQ ID NOs: 51-53; NtF3GalT=SEQ ID NOs: 54-56; CaF3GalT SEQ ID NOs: 57-59; StF3GalT SEQ ID NOs: 60-62; and S/UGT78D-B SEQ ID NOs: 63-65)

FIG. 3B. A structural model of S/UGT78D-B was generated with ColabFold89 and visualized with PyMOL2.5. The ligand position was determined according to the VvGT1 structure (World Wide Protein Data Bank). Amino acids targeted for point mutations are highlighted in green.

FIG. 3C. HPLC chromatograms showing the activity of wild-type S/UGT78D-B (BALSH) and mutated S/UGT78D-B (Bgpaq) with kaempferol and quercetin substrates, using UDP-galactose and UDP-glucose as sugar donors. The wild-type enzyme (BALSH) shows galactosyltransferase activity, but no glucosyltransferase activity, while the quadruple mutant (Bgpaq) acquires glucosyltransferase activity, producing K-3-O-glu and Q-3-O-glu. The HPLC chromatograms were obtained at a wavelength of 345 nm.

FIG. 3D. The glucosyltransferase and galactosyltransferase activities of S/UGT78D-B mutant variants. Activities are shown for quadruple, triple, double, and single mutants at positions 14, 19, 138, and 373. The presence of activity is indicated with multiple (+) marks based on product peak area in HPLC analysis (a higher number of ‘+’ marks indicate a greater level of the detected product compound), and the absence of activity is indicated with “n.d.” (not detected).

FIG. 4A-B. (A) Schematic gene structures of SIUGT78D-A and SIUGT78D-B with the CRISPR-Cas9 targeting sites and mutations in Slugt78 mutants. PSPG motif is marked on gene structures with a blue box. The guide RNA (gRNA) binding sites and the protospacer adjacent motif (PAM) are highlighted in yellow and green, respectively. The resulting deletion mutations in the mutant lines (Slugt78d-a, Slugt78d-b, and Slugt78d-a/b) are indicated. (Sequences: WT=SEQ ID NOs: 66-67; Slugt78d-a=SEQ ID NOs: 68-69; Slugt78d-b=SEQ ID NOs: 70-71; Slugt78d-a/b=SEQ ID NOs: 72-73) (B) Representative images of 3-week-old wild type (WT), Slugt78d-a, Slugt78d-b, and Slugt78d-a/b. Scale bar=3 cm.

FIG. 4C. Representative HPLC chromatogram of methanolic extracts from leaves (green traces) and pollen (brown traces) of 4-week-old wild type (WT), Slugt78d-a, Slugt78d-b, and Slugt78d-a/b mutants. The retention times for kaempferol 3-O-glucosyl (1→2) galactoside (K2), quercetin 3-O-glucosyl (1→6) rhamnoside (Q4), and kaempferol 3-O-glucosyl (1→6) rhamnoside (K4) are indicated on the chromatogram. The compound structures at the top of the graph are identified by their abbreviations: K for kaempferol, Q for quercetin, glu for glucose, gal for galactose, and rha for rhamnose.

FIG. 4D. The levels of K2, Q4 and K4 in leaves and pollen from wild type (WT) and three mutant lines. Methanolic extracts were analyzed with HPLC, and the concentration is shown in peak area per fresh weight (mAU*min FW-1). Data represent mean±SD (n=3). Statistical significance was determined using one-way ANOVA, followed by Tukey's post-hoc test, with different letters indicating significant differences among groups (P-value <0.05). Each data point is shown as an open circle on the bar graph. n.d. indicates not detected.

FIG. 4E-G. Disruption of S/UGT78D-B abolishes pollen kaempferol galactosylation, leading to defective pollen tube development and lower seed yield. (E) Cross-sectioned fruits of WT, Slugt78d-a, Slugt78d-b, and Slugt78d-a/b. (F-G) Bar graphs showing the average number of fruits per plant (F), and the number of seeds per plant (G) Data represent mean±SD (n=12 for all groups except Slugt78d-a/b, where n=3). Each data point is shown as a circle. Statistical significance was determined using one-way ANOVA, followed by Dunnett's multiple comparisons test, with adjusted P-values marked at the top.

FIG. 4H. Microscopic images of growing pollen tubes in WT, Slugt78d-a, Slugt78d-b, and Slugt78d-a/b. Red arrows indicate ruptured pollen tubes. Scale bar=200 μm.

FIG. 4I-J. (I) Violin plot showing pollen tube length for WT, Slugt78d-a, Slugt78d-b, and Slugt78d-a/b (n=200 (J) Bar graph showing the percentage of ruptured pollens. Data represent mean±SD (n=5 with each replicate containing approximately 100 pollen grains). Each data point is shown as a circle. Statistical significance was determined using one-way ANOVA, followed by Dunnett's multiple comparisons test, with adjusted P-values marked at the top.

FIG. 5A. HPLC chromatograms showing the presence of quercetin 3-O-glucosyl (1→2) galactoside (Q2) and kaempferol 3-O-glucosyl (1→2) galactoside (K2) in pollen extracts from six Solanaceae plants including Petunia hybrida (P. hybrida), Nicotiana benthamiana (N. benthamiana), Nicotiana tabacum (N. tabacum), Capsicum annuum (C. annuum), Solanum tuberosum (S. tuberosum), and Solanum lycopersicum (S. lycopersicum). The K2 peak was detected in methanolic extracts of all six Solanaceae pollens, while Q2 was detected only in petunia and two tobacco species. The HPLC chromatograms were obtained at a wavelength of 345 nm.

FIG. 5B. Conversion of flavonol monoglycosides to flavonol diglycosides in Slugt78d-b mutant pollen in chemical complementation. HPLC chromatograms show the conversion of kaempferol 3-O-galactoside (K-gal) to kaempferol 3-O-glucosyl (1→2) galactoside (K2) in the left panel, and quercetin 3-O-galactoside (Q-gal) to quercetin 3-O-glucosyl (1→2) galactoside (Q2) in the right panel, in Slugt78d-b pollen treated with 200 μM flavonol monoglycosides. Pollen treated with the same volume of DMSO (mock control) did not produce detectable levels of Q2 or K2. After flavonol monoglycoside treatment in PGM media, metabolites were extracted using a methanolic solution following 4 hours of incubation. Chromatograms were extracted at a wavelength of 345 nm, confirming the formation of flavonol diglycosides.

FIG. 5C-D. (C) Violin plot showing the pollen tube length (n=200) of Slugt78d-b pollen treated with 200 μM flavonol monoglycosides (kaempferol 3-O-galactoside, K-gal; and quercetin 3-O-galactoside, Q-gal) or an equivalent volume of DMSO (mock). Each data point is represented by a circle on the violin or bar graphs. Statistical significance was determined using one-way ANOVA followed by Dunnett's multiple comparisons test, with adjusted P-values displayed above the comparisons. (D) Violin plot showing the pollen tube length of Slugt78d-b pollen treated with varying concentrations (1 μM, 50 M, and 200 μM) of kaempferol (K) (n=200).

FIG. 5E-F. (E) Violin plot showing the pollen tube length of Slugt78d-b pollen treated with varying concentrations (1 μM, 50 μM, and 200 μM) of quercetin (Q) aglycones (n=200). (F) Bar graph illustrating the percentage of ruptured pollen tubes (n=5, with each replicate containing approximately 100 pollen grains) in Slugt78d-b pollen supplemented with 200 μM flavonols or an equivalent amount of DMSO (mock). Each data point is represented by a circle on the violin or bar graphs. Statistical significance was determined using one-way ANOVA followed by Dunnett's multiple comparisons test, with adjusted P-values displayed above the comparisons.

FIG. 6. Working model depicting the role of flavonol glycosylation in successful fertilization. (a) Organ-specific flavonol glycoside biosynthesis pathways in tomato pollen and other organs. In pollen, kaempferol (K) is synthesized from dihydrokaempferol via flavonol synthase (FLS). The glycosyltransferase S/UGT78D-B catalyzes the formation of kaempferol 3-O-galactoside (K-gal, K1), which is further modified by S/UGT79B-B to produce kaempferol 3-O-galactoside-2″-O-glucoside (K-gal-glu, K2), a pollen-specific flavonol glycoside. In other organs, kaempferol (K) and quercetin (Q) are produced via FLS, with F3′H catalyzing the conversion of dihydrokaempferol to dihydroquercetin, a precursor of quercetin. These flavonols are glycosylated by S/UGT78D-A to form kaempferol 3-O-glucoside (K-glu, K3) and quercetin 3-O-glucoside (Q-glu, Q3). Further modification by S/UGT79B-A produce kaempferol 3-O-glucosyl (1→6) rhamnoside (K-glu-rha, K4) and quercetin 3-O-glucosyl (1→6) rhamnoside (Q-glu-rha, Q4), which are abundant in vegetative tissues. (b) Schematic illustration of pollen tube growth defects in Slugt78d-b mutants and chemical complementation analysis. Wild-type (WT) pollen accumulates kaempferol (K) and its glycosylated derivatives, including K1 (kaempferol 3-O-galactoside) and K2 (kaempferol 3-O-galactoside-2″-O-glucoside), which are essential for normal pollen tube growth. In Slugt78d-b mutant pollen, the absence of K1 and K2 results in defective pollen tube growth, as observed in the mock treatment. Chemical complementation was conducted by supplementing Slugt78d-b mutant pollen with various flavonol derivatives. Among the tested compounds, only the addition of K1 successfully rescued the pollen tube growth defect by restoring K2 biosynthesis. In contrast, complementation with Q1 (quercetin 3-O-galactoside), K, or Q failed to restore normal pollen tube growth. (c) Schematic illustration of reduced seed production caused by defective pollen tube growth in Slugt78d-b mutants. In WT plants, pollen tubes successfully germinate and grow through the style, delivering male genetic material to the ovules in the ovary, resulting in normal seed production. In contrast, Slugt78d-842 b mutant pollen exhibits defective pollen tube growth, impairing fertilization and significantly reducing seed production. This schematic highlights the critical role of SIUGT78D-B in regulating pollen tube development and ensuring efficient seed production in tomato.

FIG. 7. Pollen grain morphology in wild type and three Slugt78d mutants. Microscopic images of pollen grains from wild type (WT), Slugt78d-a, Slugt78d-b, and Slugt78d-a/b plants. The images do not show any obvious morphological alterations in the pollen grains of mutants compared to wild type.

FIG. 8. Ubiquinone Q10 content in pollen grains. The levels of free kaempferol in the pollen grains of WT 101 and three Slugt78d mutants. Data represent mean±SD (n=3). Statistical significance was determined using one-way ANOVA, followed by Tukey's post-hoc test, with different letters indicating significant differences among groups (P-value <0.05). The metabolite levels were normalized to fresh weight (FW). Each data point is shown as an open circle on the bar graph.

### DETAILED DESCRIPTION

### I. Definitions:

Unless otherwise defined, all terms of art, notations, and other scientific terminology used herein are intended to have the meanings commonly understood by those of skill in the art to which this invention pertains. Some techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y.; Current Protocols in Molecular Biology (Ausbel et al., eds., John Wiley & Sons, Inc. 2001; Transgenic Plants: Methods and Protocols (Leandro Pena, ed., Humana Press, 1st edition, 2004); and, Agrobacterium Protocols (Wan, ed., Humana Press, 2nd edition, 2006). As appropriate, procedures involving the use of commercially available kits and reagents are generally carried out in accordance with manufacturer defined protocols and/or parameters unless otherwise noted.

The use of “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

The term “about” or “approximately” indicates within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of 0 to 20%, 0 to 10%, 0 to 5%, or up to 1% of a given value. Where particular values are described in the application and claims, unless otherwise stated the term “about” means within an acceptable error range for the particular value should be assumed.

All ranges are to be interpreted as encompassing the endpoints in the absence of express exclusions such as “not including the endpoints”; thus, for example, “within 10-15” includes the values 10 and 15. One skilled in the art will understand that the recited ranges include the end values, as well as whole numbers in between the end values, and where practical, rational numbers within the range (e.g., the range 5-10 includes 5, 6, 7, 8, 9, and 10, and where practical, values such as 6.8, 9.35, etc.). When values are expressed as approximations, by use of the antecedent “about,” it will be understood that the particular value forms a further aspect. For example, if the value “about 10” is disclosed, then “10” is also disclosed.

The term “nucleic acid” refers to deoxyribonucleotides or ribonucleotides and polymers thereof (“polynucleotides”) in either single- or double-stranded form. Unless specifically limited, the term polynucleotide encompasses nucleic acids containing known analogues of natural nucleotides which have similar binding properties as the reference nucleic acid and are metabolized in a manner similar to naturally occurring nucleotides. Unless specifically limited, the term polynucleotide encompasses nucleic acids having one or more modified nucleotides. Modified nucleotides can modify binding properties or alter in vitro or in vivo stability. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991, Nucleic Acid Res. 19:5081; Ohtsuka et al., 1985 J. Biol. Chem. 260:2605-2608; and Cassol et al., 1992; Rossolini et al., 1994, Mol. Cell. Probes 8:91-98). The term nucleic acid is used interchangeably with gene, cDNA, and mRNA encoded by a gene.

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, or 95% identity over a specified region, when compared and aligned for maximum correspondence over a comparison window, or designated region as measured using a sequence comparison algorithms, or by manual alignment and visual inspection.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

The term “plant” includes whole plants, plant organs (e.g., leaves, stems, flowers, roots, reproductive organs, embryos, and parts thereof, etc.), seedlings, seeds, plant cells, and progeny thereof. The class of plants which can be used in the method of the invention is generally as broad as the class of higher plants amenable to transformation techniques, including angiosperms (monocotyledonous and dicotyledonous plants), as well as gymnosperms. It includes plants of a variety of ploidy levels, including polyploid (e.g., octoploid), diploid, haploid, and hemizygous.

The term “locus” refers to a position in the genome that corresponds to a measurable characteristic (e.g., a trait) or gene. A locus can be a genomic region or section of DNA (the locus) which correlates with a variation in a phenotype. A locus can comprise a single or multiple genes or other genetic information within a contiguous genomic region or linkage group.

A “marker” or “genetic marker” refers to a gene or nucleotide sequence that can be used to identify the presence or location of a trait determinant, locus, gene, and/or allele. A genetic marker may be described as a variation at a given genomic locus. A genetic marker may be a short DNA sequence, such as a sequence surrounding a single base-pair change (for example as in a single nucleotide polymorphism (SNP)), or a longer DNA sequence, for example, a microsatellite/simple sequence repeat (SSR)). A “marker allele” refers to the version of the marker that is present in a particular individual. A genetic marker can be used to identify individuals, genes, or loci in an off-spring originating from an individual parent. An “UGT78D-B marker” is a genetic marker that is linked, closely linked, tightly linked, or extremely tightly linked to an UGT78D-B gene.

“Linked” refers to one or more genes or markers that are located within about 65 megabases (Mb) of one another on the same chromosome. Thus, two “linked” genes or markers may be separated, for example, by about 65 Mb, about 60 Mb, about 55 Mb, about 50 Mb, about 45 Mb, about 40 Mb, about 35 Mb, about 30 Mb, about 25 Mb, about 20 Mb, about 15 Mb, about 10 Mb, about 9.0. Mb, about 8.0 Mb, about 7.0 Mb, about 6.0 Mb, about 5.2 Mb, about 4.0 Mb, about 3.0 Mb, about 2.0 Mb, about 1.0 Mb, or fewer Mb.

“Introgression” or “introgressing” of a UGT78D-B locus means introduction of a UGT78D-B locus from a donor plant into a recipient plant by standard breeding techniques, wherein selection can be done phenotypically or with the use of a genetic marker linked to the UGT78D-B locus markers through marker-assisted breeding, or combinations of these. The process of introgressing is often referred to as “backcrossing” when the process is repeated two or more times. In introgressing or backcrossing, the “donor” parent refers to the parental plant with the desired gene or locus to be introgressed. The “recipient” parent (used one or more times) or “recurrent” parent (used two or more times) refers to the parental plant into which the gene or locus is being introgressed. Selection is started in the F1 or any further generation from a cross between the recipient plant and the donor plant, suitably by using markers. The skilled person is however familiar with creating and using new molecular markers that can identify or are linked to the UGT78D-Blocus.

A “homolog” or “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologs (orthologous sequences) and paralogs (paralogous sequences). Homologous genes, for example, typically descend from a common ancestral DNA sequence, either through a speciation event (orthologous genes) or a genetic duplication event (paralogous genes). “Orthologs” are genes and products thereof in different species that evolved from a common ancestral gene by speciation and retain the same or similar function. An ortholog is a gene that is related by vertical descent and is responsible for substantially the same or identical functions in different organisms. “Paralogous” genes include genes related by duplication within a genome. Paralogs can evolve new functions in the course of evolution.

A “heterologous” sequence is a sequence which is not normally present in a cell, genome, or gene in the genetic context in which the sequence is currently found. A heterologous sequence can be a sequence derived from the same gene and/or cell type, but introduced into the cell or a similar cell in a different context, such as on an expression vector or in a different chromosomal location or with a different promoter. A heterologous sequence can be a sequence derived from a different gene or species than a reference gene or species. A heterologous sequence can be from a homologous gene from a different species, from a different gene in the same species, or from a different gene from a different species. For example, a regulatory sequence may be heterologous in that it is linked to a different coding sequence relative to the native regulatory sequence.

Compositions or methods “comprising” or “including” one or more recited elements may include other elements not specifically recited. For example, a composition that “comprises” or “includes” a marker may contain the marker alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which it does not.

The term “and/or” refers to and encompasses any and all possible combinations of one or more of the associated listed items, as well as the lack of combinations when interpreted in the alternative (“or”). The term “or” refers to any one member of a particular list and also includes any combination of members of that list.

The singular forms of the articles “a,” “an,” and “the” include plural references unless the context clearly dictates otherwise. For example, the term “a marker” or “at least one marker” can include a plurality of markers, including mixtures thereof.

An “RNA-guided DNA endonuclease” is an enzyme (endonuclease) that uses RNA-DNA complementarity to identify target sites for sequence-specific double-stranded DNA (dsDNA) cleavage. An RNA-guided DNA endonuclease may be, but is not limited to, a zCas9 nuclease, a Cas9 nuclease, type II Cas nuclease, an nCas9 nuclease, a type V Cas nuclease, a Cas12a nuclease, a Cas12b nuclease, a Cas12c nuclease, a CasY nuclease, a CasX nuclease, a Cas12i nuclease, or an engineered RNA-guided DNA endonuclease.

A “guide RNA” (gRNA) comprises an RNA sequence (tracrRNA) bound by Cas and a spacer sequence (crRNA) that hybridizes to a target sequence and defines the genomic target to be modified. The tracrRNA and crRNA may be linked to form a “single chimeric guide RNA” (sgRNA).

The term “CRISPR RNA (crRNA)” has been described in the art (e.g., in Makarova et al. (2011) Nat Rev Microbiol 9:467-477; Makarova et al. (2011) Biol Direct 6:38; Bhaya et al. (2011) Annu Rev Genet 45:273-297; Barrangou et al. (2012) Annu Rev Food Sci Technol 3:143-162; Jinek et al. (2012) Science 337:816-821; Cong et al. (2013) Science 339:819-823; Mali et al. (2013) Science 339:823-826; and Hwang et al. (2013) Nature Biotechnol 31:227-229). A crRNA contains a sequence (spacer sequence or guide sequence) that hybridizes to a target sequence in the genome. A target sequence can be any sequence that is unique compared to the rest of the genome and is adjacent to a protospacer-adjacent motif (PAM).

A “protospacer-adjacent motif” (PAM) is a short sequence recognized by the CRISPR complex. The precise sequence and length requirements for the PAM differ depending on the CRISPR system used, but PAMs are typically 2-5 base pair sequences adjacent the protospacer (i.e., target sequence). Non-limiting examples of PAMs include NGG, NNGRRT, NN[A/C/T]RRT, NGAN, NGCG, NGAG, NGNG, NGC, and NGA.

A “trans-activating CRISPR RNA” (tracrRNA) is an RNA species facilitates binding of the RNA-guided DNA endonuclease (e.g., Cas) to the guide RNA.

A “CRISPR system” comprises a guide RNA, either as a crRNA and a tracrRNA (dual guide RNA) or an sgRNA, and a RNA-guided DNA endonuclease. The guide RNA directs sequence-specific binding of the RNA-guided DNA endonuclease to a target sequence. In some embodiments, the RNA-guided DNA endonuclease contains a nuclear localization sequence. In some embodiments, the CRISPR system further comprises one or more fluorescent proteins and/or one or more endosomal escape agents. In some embodiments, the gRNA and RNA-guided DNA endonuclease are provided in a complex. In some embodiments, the gRNA and RNA-guided DNA endonuclease are provided in one or more expression constructs (CRISPR constructs) encoding the gRNA and the RNA-guided DNA endonuclease. Delivery of the CRISPR construct(s) to a cell results in expression of the gRNA and RNA-guided DNA endonuclease in the cell. The CRISPR system can be, but is not limited to, a CRISPR class 1 system, a CRISPR class 2 system, a CRISPR/Cas system, a CRISPR/Cas9 system, a CRISPR/zCas9 system and a CRISPR/Cas3 system.

A “DNA donor template” is a nucleic acid, such as a single stranded DNA, linear double strand DNA, plasmid DNA or AAV sequence that provides the homology necessary for precise repair of a double-strand break. A DNA donor template contains two regions of homology, one region of homology to either side of the double strand break. A DNA donor template can also contain a heterologous sequence between the two regions of homology. Each region of homology can be about 30 to about 100 nucleotides in length.

A “regenerant” is a plant produced from a plant tissue cell, such as a genetically modified plant tissue cell.

“Male fertility” refers to the ability of a plant to generate functional anthers and pollen, and to form functional pollen tubes. A deficiency in one or more of: generating anthers, generating pollen, or pollen tube growth can decrease male fertility.

UGT78D-B is a flavonol (kaempferol) 3-O-galactosyltransferase (also termed F3Gal). A UGT78D-B catalyzes attachment of a galactosyl group to the 3 position of a kaempferol molecule to form kaempferol 3-O-galactoside. In tomato, the S/UGT78D-B protein has the amino acid sequence of SEQ ID NO:3. UGT78D-B protein proteins in other plant species include, but are not limited to, NtF3Gal (amino acid sequence provided in SEQ ID NO:5), CaF3Gal (amino acid sequence provided in SEQ ID NO:7), and StF3Gal (amino acid sequence provided in SEQ ID NO: 9). NtF3Gal, CaF3Gal, and StF3Gal are orthologs of S/UGT78D-B. Additional UGT78D-B (F3Gal) proteins in other plant species (i.e., orthologs of S/UGT78D-B) can be identified by sequence analysis methods available in the art and by biochemical assays for attachment of a galactosyl group to the 3 position of a kaempferol molecule to form kaempferol 3-O-galactoside such as those described herein (see examples 2 and 6). In some embodiments, an ortholog of SIUGT78D-B comprises an amino acid sequence having at least 70%, at least 75%, at least 80%, at least 85%, at least 90%, or at least 95% identity to SEQ ID NO:3 and facilitates or catalyzes attachment of a galactosyl group to the 3 position of a kaempferol molecule to form kaempferol 3-O-galactoside.

The UGT78D-B gene encodes the UGT78D-B protein. In tomato, S/UGT78D-B is encoded by the SIUGT78D-B gene (see SEQ ID NOs: 1 and 2 for ORF and mRNA SIUGT78D-B sequences). In tomato, the SIUGT78D-B gene encodes an mRNA having the nucleic acid sequence of SEQ ID NO:2 (or encodes an ORF having the nucleic acid sequence of SEQ ID NO:1). UGT78D-B genes in other plant species include, but are not limited to, NtF3Gal (encoding an ORF having the nucleic acid sequence provided in SEQ ID NO:4), CaF3Gal (encoding an ORF having the nucleic acid sequence provided SEQ ID NO:6), and StF3Gal (encoding an ORF having the nucleic acid sequence provided in SEQ ID NO:8). NtF3Gal, CaF3Gal, and StF3Gal are orthologs of SIUGT78D-B. Additional UGT78D-B (F3Gal) genes in other plant species (i.e., orthologs of SIUGT78D-B) can be identified by sequence analysis methods available in the art and by biochemical assays of the encoded protein for attachment of a galactosyl group to the 3 position of a kaempferol molecule to form kaempferol 3-O-galactoside such as those described herein (see examples 2 and 6). In some embodiments, an ortholog of the S/UGT78D-B gene comprises a nucleic acid sequence encoding a protein having an amino acid sequence that is at least 70%, at least 75%, at least 80%, at least 85%, at least 90%, or at least 95% identity to SEQ ID NO:3 and facilitates or catalyzes attachment of a galactosyl group to the 3 position of a kaempferol molecule to form kaempferol 3-O-galactoside.

### II. Overview

We show that the UGT78D-B protein is produced primarily in pollen and catalyzes attachment of a galactosyl group to the 3 position of a kaempferol molecule to form kaempferol 3-O-galactoside. In the absence of kaempferol 3-O-galactoside, plants are unable to generate kaempferol 3-O-glucosyl (1→2) galactoside (termed K-gal-glu or K2), which is necessary for proper pollen tube growth and successful seed production. We further shown that UGT78D-B is necessary for proper pollen tube growth and male fertility. Genetically modified plants having a disrupted UGT78D-B gene have decreased or defective pollen tube growth and markedly reduced seed set (i.e., reduced male fertility).

Described are methods and compositions for modifying male fertility in a plant comprising: modifying or reducing the activity of a UGT78D-B (flavonol (kaempferol) 3-O-galactosyltransferase). Methods disclosed herein can produce male sterile plants by reducing expression of an endogenous UGT78D-B gene or modifying activity of the encoded UGT78D-B in plants. As used herein a UGT78D-B comprises a Solanum lycopersicum UGT78D-B (SEQ ID NO: 3) or an ortholog thereof in another plant species (e.g., SEQ ID NO:5, 7, or 9). As used herein a UGT78D-B genomic locus comprises a Solanum lycopersicum UGT78D-B genomic locus or an ortholog thereof in another plant species. As used herein a UGT78D-B gene comprises a Solanum lycopersicum UGT78D-B gene (mRNA comprises SEQ ID NO:1 or 3) or an ortholog thereof in another plant species (e.g., mRNA comprising SEQ ID NO:4, 6, or 8).

Also described are methods of restoring male fertility in plants by providing a wild-type UGT78D-B gene, or repairing a loss of function mutation in a genomic UGT78D-B locus, in a plant in which endogenous UGT78D-B gene contains a mutation that decreases expression or decreases activity of the encoded UGT78D-B rhomboid protease. Fertility in plants can be generated by restoring UGT78D-B function of a male sterile plant for a two-line breeding system.

Male fertility in a plant in which the endogenous UGT78D-B gene contains a mutation that decreases expression or decreases activity of the encoded UGT78D-B can also be restored by contacting or supplementing the plant with a kaempferol 3-O-galactoside.

The plant can be a diploid species, a tetraploid species, a hexaploid species, an octoploid species, or any other ploidy species or hybrid thereof. The plant can be, but is not limited to, Solanaceae plant, other dicot plants, or a monocot plant. A Solanaceae plant can be, but is not limited to, a tomato plant (Solanum lycopersicum), a tobacco plant (Nicotiana tabacum), a pepper plant (Capsicum annuum), or a potato plant (Solanum tuberosum). Other dicot plants include, but are not limited to, a blackberry plant, a soybean plant, a canola plant, an alfalfa plant, a sunflower plant, a cotton plant, a peanut plant, a strawberry plant, a blueberry plant, and a stone fruit (drupe) plant.

The monocot plant can be, but is not limited to, a rice plant (Oryza sativa), a maize (corn) plant, a sorghum plant, a rye plant, a barley plant, a wheat plant, an oat plant, or a sugarcane plant.

Described are constructs and systems for reducing expression of the UGT78D-B gene in a plant to induce male sterility. Also described are constructs and systems for genetically modifying a UGT78D-B genomic locus in a plant to reduce expression of the UGT78D-B gene or to decrease activity of UGT78D-B.

In some embodiments, genetically modifying a plant comprises introducing into the plant an RNA interference (RNAi) molecule or an antisense nucleotide targeting the UGT78D-B mRNA or expressing in the plant an RNAi nucleic acid sequence or an antisense nucleic acid sequence that targets the UGT78D-B mRNA to inhibit or knock down expression of the UGT78D-B mRNA. Antisense and RNAi molecules can be made using methods available in the art.

In some embodiments, genetically modifying a plant comprises creating a deletion, insertion, substitution, frameshift, or nonsense mutation, or combinations thereof, in the UGT78D-B gene wherein the mutant UGT78D-B encoded by the modified UGT78D-B gene has a reduced ability catalyze attachment of a galactosyl group to the 3 position of a kaempferol molecule.

In some embodiments, genetically modifying a plant comprises modifying an endogenous UGT78D-B genomic locus to reduce or knock our expression of the UGT78D-B gene.

In some embodiments, constructs and systems for CRISPR/Cas-mediated modification of a UGT78D-B genomic locus in a plant are described. The constructs and systems can be used to induce male sterility (e.g., in a plant having a wild type UGT78D-B gene) or restore male fertility (e.g., in a plant having a modified ugt78d-b gene) in a plant.

In some embodiments, nucleic acids for modifying male fertility in a plant are described. The nucleic acids include, but are not limited to, nucleic acids comprising crRNAs or gRNAs and nucleic acids encoding crRNAs or gRNAs.

In some embodiments, nucleic acids for modifying male fertility in plants using CRISPR systems are described. The nucleic acids include, but are not limited to, nucleic acids comprising crRNAs or gRNAs, nucleic acids encoding crRNAs or gRNAs, and DNA donor template nucleic acids.

The CRISPR systems can be used to modify an endogenous wild-type UGT78D-B genomic locus or a mutant UGT78D-B gene or genomic locus. The nucleic acids include, but are not limited to, nucleic acids comprising crRNAs or gRNAs, nucleic acids encoding crRNAs or gRNAs, and DNA donor template nucleic acids.

In some embodiments, a CRISPR system as described herein, for use in modifying a SIUGT78D-B genomic locus in a tomato plant comprises a guide RNA comprising SEQ ID NO:10. In some embodiments, a CRISPR system as described herein, for use in modifying a NtF3Gal genomic locus in a tobacco plant comprises a guide RNA comprising SEQ ID NO:11 or 12. In some embodiments, a CRISPR system as described herein, for use in modifying a CaF3Gal genomic locus in a pepper plant comprises a guide RNA comprising SEQ ID NO:13 or 14. In some embodiments, a CRISPR system as described herein, for use in modifying a StF3Gal genomic locus in a potato plant comprises a guide RNA comprising SEQ ID NO: 15 or 16. Guide RNAs for CRISPR systems for use in modifying orthologs of SIUGT78D-B in other plants can be formed using methods available in the art for designing guide RNAs.

In some embodiments, the described CRISPR systems are used to modify an endogenous UGT78D-B genomic locus in a plant, and the resulting plants have a reduced ability to catalyze attachment of a galactosyl group to the 3 position of a kaempferol molecule to form kaempferol 3-O-galactoside. In some embodiments, the modified plants have reduced male fertility. Mail fertility in the modified plants can be reduced by at least 25%, at least 30%, at least 35%, at least 40%, at least 45%, at least 50%, at least 60%, at least 65%, at least 70%, at least 75%, at least 80%, at least 85%, at least 90%, or at least 95% relative to a similar plant that does not contain a modified UGT78D-B gene. In some embodiments, the modified plants have reduced pollen tube growth. Pollen tube growth in the modified plants can be reduced by at least 25%, at least 30%, at least 35%, at least 40%, at least 45%, at least 50%, at least 60%, at least 65%, at least 70%, at least 75%, at least 80%, at least 85%, at least 90%, or at least 95% relative to a similar plant that does not contain a modified UGT78D-B gene. In some embodiments, the modified plants have reduced seed setting. Seed setting in the modified plants can be reduced by at least 25%, at least 30%, at least 35%, at least 40%, at least 45%, at least 50%, at least 60%, at least 65%, at least 70%, at least 75%, at least 80%, at least 85%, at least 90%, or at least 95% relative to a similar plant that does not contain a modified UGT78D-B gene. Pollen tube grown is reduced for pollen originating from the ugt78d-b plant (i.e., a plant having a disruption of the UGT78D-B gene. The genotype of the pistil does not significantly influence pollen tube growth if pollen is from a ugt78d-b plant.

Also described are CRISPR constructs for repairing a mutant UGT78D-B gene or UGT78D-B genomic locus in a plant.

CRISPR systems for modifying an endogenous wild-type or mutant gene or gene locus are readily designed using the methods available in the art and the information described herein.

In some embodiments, plants having a reduced male fertility phenotype produced using any one or more of the described CRISPR constructs are described. The described UGT78D-B genomic locus can be targeted to genetically modify plants to yield a reduced male fertility phenotype.

In some embodiments, fertility is restored to a plant having a reduced male fertility phenotype caused by loss of UGT78D-B function by introducing a heterologous UGT78D-B gene encoding a functional UGT78D-B (e.g., capable of catalyzing attachment of a galactosyl group to the 3 position of a kaempferol molecule to form kaempferol 3-O-galactoside). In some embodiments, fertility is restored to a plant having a reduced male fertility phenotype caused by loss of UGT78D-B function by repairing a mutation in an endogenous UGT78D-B gene, e.g., via CRISPR mediated gene repair.

In some embodiments, fertility is restored to a plant having a reduced male fertility phenotype caused by loss of UGT78D-B function by contacting or supplementing the plant or pollen with kaempferol 3-O-galactoside or kaempferol 3-O-glucosyl (1→2) galactoside.

### III. Targeted Genetic Modification

A targeted genetic modification can comprise a targeted alteration to a polynucleotide of interest including, for example, a UGT78D-B genomic locus, or insertion of a heterologous gene in a location suitable for expression of a heterologous gene in a plant. Such targeted modifications include, but are not limited to, additions of one or more nucleotides, deletions of one or more nucleotides, substitutions of one or more nucleotides, a knockout of the polynucleotide of interest or a portion thereof, and a knock-in of a polynucleotide of interest or a portion thereof (e.g., a heterologous UGT78D-B gene, coding sequence, or fragment thereof). In certain embodiments, at least 1, 2, 3, 4, 5, 7, 8, 9, 10 or more nucleotides are changed to form the targeted genomic modification.

Various methods can be used to generate the targeted modification in the polynucleotide or plant genome of interest. Methods of obtaining the targeted genetic modifications include, but are not limited to, unaided homologous recombination, recombinase-based insertion, and DNA repair-based insertion and are other methods known in the art (as described, in e.g., Dong et al., 2021, PNAS. 118 (22) e2004834117). Recombinase-based insertion can comprise systems and constructs involving site-specific recombinases, including but not limited to, Cre:loxP systems, Flp:FRT systems, Dre:rox systems, VCre:loxV systems, Gin: gix systems, Bxb1:attP/attB systems, and phiC31:attP/attB systems. DNA repair-based insertion methods rely upon the activity of a nuclease agent.

The term “recognition site for a nuclease agent” includes a DNA sequence at which a nick or double-strand break is induced by a nuclease agent. The recognition site for a nuclease agent can be endogenous (or native) to the cell or the recognition site can be exogenous to the cell.

The length of the recognition site can vary, and includes, for example, recognition sites that are about 30-36 bp for a zinc finger nuclease (ZFN) pair (i.e., about 15-18 bp for each ZFN), about 36 bp for a Transcription Activator-Like Effector Nuclease (TALEN), or about 20 bp for a CRISPR/Cas9 guide RNA.

In some embodiments, each monomer of the nuclease agent recognizes a recognition site of at least 9 nucleotides. In some embodiments, the recognition site is from about 9 to about 12 nucleotides in length, from about 12 to about 15 nucleotides in length, from about 15 to about 18 nucleotides in length, or from about 18 to about 21 nucleotides in length, and any combination of such subranges (e.g., 9-18 nucleotides). It is recognized that a given nuclease agent can bind the recognition site and cleave that binding site or alternatively, the nuclease agent can bind to a sequence that is different from the recognition site. Moreover, the term recognition site comprises both the nuclease agent binding site and the nick/cleavage site irrespective whether the nick/cleavage site is within or outside the nuclease agent binding site. In another variation, the cleavage by the nuclease agent can occur at nucleotide positions immediately opposite each other to produce a blunt end cut or, in other cases, the incisions can be staggered to produce single-stranded overhangs, also called “sticky ends,” which can be either 5′ overhangs, or 3′ overhangs.

Any nuclease agent that induces a nick or double-strand break into a desired recognition site can be used in the methods and compositions disclosed herein. A naturally occurring or native nuclease agent can be employed so long as the nuclease agent induces a nick or double-strand break in a desired recognition site. Alternatively, a modified or engineered nuclease agent can be employed. An “engineered nuclease agent” includes a nuclease that is engineered (modified or derived) from its native form to specifically recognize and induce a nick or double-strand break in the desired recognition site. Producing a nick or double-strand break in a recognition site or other DNA can be referred to herein as “cutting” or “cleaving” the recognition site or other DNA. Assays to measure the double-strand break of a recognition site by a nuclease agent are known in the art (e.g., TaqMan® qPCR assay, Frendewey D. et al., Methods in Enzymology, 2010, 476:295-307, which is incorporated by reference herein in its entirety).

In some embodiments, the recognition site is positioned within a target genomic locus (e.g., a UGT78D-B genomic locus). Such a position can be located within a coding region of the target genomic locus, a regulatory region (e.g., a promoter or enhancer region), or an intron. In specific embodiments, a nick or double-strand break at the recognition site disrupts the activity of the target gene.

In some embodiments, the nuclease agent is a Transcription Activator-Like Effector Nuclease (TALEN). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See e.g., WO 2010/079430, which is herein incorporated by reference. Examples of suitable TAL nucleases, and methods for preparing suitable TAL nucleases, are disclosed, e.g., in US20110239315, US20110269234, US20110145940, US20030232410, US20050208489, US20050026157, US20050064474, US20060188987, and US20060063231, each of which is incorporated herein by reference.

In some embodiments, the nuclease agent employed in the various methods and compositions disclosed herein comprise a zinc-finger nuclease (ZFN). Exemplary ZFNs are described in, e.g., US20060246567, US20080182332, US20020081614, US20030021776, WO2002057308, US20130123484, US20100291048, and WO2011017293, each of which is herein incorporated by reference.

In some embodiments, the nuclease agent comprises a meganuclease. Meganucleases include LAGLIDADG, GIY-YIG, H-N-H, and His-Cys box families. Exemplary meganuclease are described in, e.g., WO2005105989, WO2003078619, WO2006097854, WO2006097853, WO2006097784, and WO2004031346, each of which is herein incorporated by reference.

The nuclease agent employed in the various methods and compositions can also comprise a CRISPR/Cas system. Described are nucleic acids for producing AFR and/or ARN resistant plants using a CRISPR (e.g., CRISPR/Cas) system. The described nucleic acids can be used to target modification of the FaRCa1 locus and/or to insert or express one or more AFR and/or ARN resistance genes in a plant.

A CRISPR system comprises an RNA-guided DNA endonuclease enzyme and a CRISPR RNA. In some embodiments, a CRISPR RNA is part of a guide RNA. In some embodiments, the RNA-guided DNA endonuclease enzyme is a Cas9 protein. In some embodiments, a CRISPR system comprises one or more nucleic acids encoding an RNA-guided DNA endonuclease enzyme (such as, but not limited to a Cas9 protein) and a guide RNA. A guide RNA can comprise a CRISPR RNA (crRNA) and a trans-activating CRISPR RNA (tracrRNA), either as separate molecules or a single chimeric guide RNA (sgRNA). The guide RNA contains a guide sequence having complementarity to a sequence in the target gene genomic region. The Cas protein can be introduced into the plant in the form of a protein or a nucleic acid (DNA or RNA) encoding the Cas protein (e.g., operably linked to a promoter expressible in the plant). The guide RNA can be introduced into the plant in the form of RNA or a DNA encoding the guide RNA (e.g., operably linked to a promoter expressible in the plant). In some embodiments, the CRISPR system further includes a DNA donor template. In some embodiments, the CRISPR system can be delivered to a plant or plant cell via a bacterium. The bacterium can be, but is not limited to, Agrobacterium tumefaciens.

In some embodiments, the CRISPR system is designed to knockout a UGT78D-B genomic locus, coding sequence, or fragment thereof, thereby reducing male fertility. In some embodiments, the CRISPR system is designed insert a heterologous UGT78D-B gene, coding sequence, or fragment thereof into the genome thereby resulting in restoration of male fertility.

In some embodiments, the CRISPR system is designed to introduce a nutation into a UGT78D-B genomic locus, wherein the mutation reduces or eliminates activity of the encoded UGT78D-B protein, thereby reducing male fertility. In some embodiments, the CRISPR system is designed introduce a mutation into a mutant UGT78D-B gene that encodes a UGT78D-B protein having reduced or no activity, wherein the mutation results in increased or restored activity of the encoded UGT78D-B protein, thereby resulting in restoration of male fertility.

The CRISPR/Cas system can be, but is not limited to, a CRISPR class 1 system, CRISPR class 2 system, CRISPR/Cas system, a CRISPR/Cas9 system, a CRISPR/zCas9 system or CRISPR/Cas3 system.

Guide sequences suitable for forming gRNAs or crRNAs for CRISPR system mediated genetic modification of a UGT78D-Blocus are described. Suitable guide sequences include 17-20 nucleotide sequences along the UGT78D-B locus, that are unique compared to the rest of the genome and immediately adjacent (5′) to a protospacer-adjacent motif (PAM) site. For the RNA-guided DNA endonuclease enzyme zCas9, a PAM site is NGG. Thus, any unique 17-20 nucleotide sequence immediately 5′ of a 5′-NGG-3′ the UGT78D-B locus can be used in forming a gRNA. In some embodiments, the guide sequence is 100% complementary to the target sequence. In some embodiments, the guide sequence is at least 90% or at least 95% complementary to the target sequence. In some embodiments, the guide sequence contains 0, 1, or 2 mismatches when hybridized to the target sequence. In some embodiments, a mismatch, if present, is located distal to the PAM, in the 5′ end of the guide sequence. In some embodiments, the gRNA comprises a sequence identical to or complementary to a sequence present in a UGT78D-B genomic locus. In some embodiments, the gRNA comprises a sequence identical to or complementary to a 17-20 nucleotide sequence present in a UGT78D-B genomic locus. In some embodiments, the gRNA comprises a sequence identical to or complementary to a sequence present in any of SEQ ID NOs: 1, 2, 4, 6, or 8. In some embodiments, the gRNA comprises a sequence identical to or complementary to a 17-20 nucleotide sequence present in any of SEQ ID NOs: 1, 2, 4, 6, or 8. In some embodiments, the gRNA comprises any one of SEQ ID NOs: 10-16.

Additional guide sequence suitable for forming gRNAs or crRNAs for CRISPR system mediated genetic modification of plants include 17-20 nucleotide sequences in the genome that are unique compared to the rest of the genome and immediately adjacent (5′) to a protospacer-adjacent motif (PAM) site and suitable for insertion of a heterologous gene. Such sites are term safe harbor sites.

The DNA donor template contains sequence to be inserted into the genome of the plant. In some embodiments, the DNA donor template to be used with a CRISPR system comprises coding sequences for a heterologous UGT78D-B gene. The donor template can further comprise one or more regulatory sequences operatively linked to the coding sequence that drives expression of the coding sequence in the plant cell. In some embodiments, the DNA donor template to be used with a CRISPR system comprises sequence for modifying the endogenous UGT78D-B genomic locus to reduce or knock out expression of the UGT78D-B gene or to modify the activity of the encoded UGT78D-B. In some embodiments, the CRISPR system introduces a mutation into the genomic UGT78D-B coding sequence that results in loss of enzymatic activity of the encoded UGT78D-B. In some embodiments, the mutation comprises a deletion, insertion, substitution, or frameshift mutation. In some embodiments, the mutation comprises a deletion of 2-22 nucleotides in the UGT78D-B gene locus near the target site of the gRNA.

DNA donor templates further comprise 5′ and 3′ homology regions located 5′ and 3′ to the sequence to be inserted into the genome. The homology regions comprise about 30 to about 100 nucleotides that are complementary to a corresponding number of nucleotides in the genome on either side of the double strand break created by the CRISPR nuclease.

The DNA donor template can be provided a single strand DNA, double strand DNA, plasmid DNA, or viral vector DNA (e.g., adeno-associated vector DNA).

It is understood that RNA equivalents of any listed DNA sequences, substituting uracils (U) for thymines (T), may be used. An “RNA equivalent” is an RNA molecule having essentially the same complementary base pair hybridization properties as the listed DNA sequence.

CRISPR modification of a UGT78D-B genomic locus or other genomic site is not limited to the CRISPR/zCas9 system. Other CRISPR systems using different nucleases and having different PAM sequence requirements are known in the art. PAM sequences vary by the species of RNA-guided DNA endonuclease. For example, Class 2 CRISPR-Cas type II endonuclease derived from S. pyogenes utilizes an NGG PAM sequence located on the immediate 3′ end of the guide sequence. Other PAM sequences include, but are not limited to, NNNNGATT (Neisseria meningitidis), NNAGAA (Streptococcus thermophilus), and NAAAAC (Treponema denticola). Guide sequences for CRISPR systems having nucleases with different PAM sequence requirements are identified as described above for zCas9, substituting the different PAM sequences.

Two or more guide RNAs can used with the same RNA-guided DNA endonuclease (e.g., Cas nuclease) or different RNA-guided DNA endonucleases.

Any of the above-described guide RNAs can be provided as an RNA or a DNA encoding the RNA.

In some embodiments, a CRISPR system comprises one or more guide RNAs and a nucleic acid encoding an RNA-guided DNA endonuclease. In some embodiments, a CRISPR system comprises one or more guide RNAs, a nucleic acid encoding an RNA-guided DNA endonuclease, and a DNA donor template.

In some embodiments, a CRISPR system comprises one or more guide RNAs and one or more nucleic acids encoding two or more different RNA-guided DNA endonucleases. In some embodiments, a CRISPR system comprises one or more guide RNAs, one or more nucleic acids encoding two or more different RNA-guided DNA endonucleases, and at least one DNA donor template.

In some embodiments, a CRISPR system comprises a guide RNA and an RNA-guided DNA endonuclease in a complex. In some embodiments, a CRISPR system comprises a guide two or more RNAs each in a complex with an RNA-guided DNA endonuclease. In some embodiments, a CRISPR system comprises a guide RNA and an RNA-guided DNA endonuclease in a complex and a DNA donor template.

### IV. Modified Plants

Described are genetically modified plants comprising a modified UGT78D-B gene or genomic locus. Also described are methods of making the genetically modified plants and plants using the described methods. In some embodiments, the plants have reduced male fertility (i.e., male sterility). In some embodiments, the methods comprise using a CRISPR system to genetically modify an endogenous UGT78D-B gene or genomic locus. Thus, methods of reducing male fertility, or introducing male sterility, in a plant are described. Also described are methods for restoring male fertility in a plant in which the endogenous UGT78D-B gene or genomic locus was previously modified. In some embodiments, CRISPR is used to genetically modify the plant. Other methods known in the art may also be used to modify an endogenous UGT78D-B genomic locus.

Described are methods of generating genetically modified male sterile or fertile male plants comprising introducing into a plant, a plant tissue, or a plant cell, one or more of the described CRISPR systems to modify or insert a UGT78D-B gene or sequence. In some embodiments, genetically modified male sterile plants created using a CRISPR system are described. In some embodiments, the CRISPR system is a CRISPR/Cas system.

In some embodiments, methods are described for producing a male sterile or fertile male plant, the methods comprising the step of introducing into the plant one or more of the described CRISPR systems.

Nucleic acids may be introduced into a plant cell or cells using a number of methods known in the art, including but not limited to electroporation, DNA bombardment or biolistic approaches, microinjection, via the use of various DNA-based vectors such as Agrobacterium tumefaciens and Agrobacterium rhizogenes vectors, and CRISPR or CRISPR/Cas9. Various methods for introducing the transgene expression vector constructs of the invention into a plant or plant cell are well known to those skilled in the art, and any method capable of transforming the target plant or plant cell may be utilized. Once a plant cell has been successfully transformed, it may be cultivated to regenerate a transgenic plant (regenerant).

In some embodiments, Agrobacterium tumefaciens is used to deliver CRISPR system nucleic acids to a plant. Agrobacterium-mediated transformation of a large number of plants are extensively described in the literature (see, for example, Agrobacterium Protocols, Wan, ed., Humana Press, 2^{nd} edition, 2006). Various methods for introducing DNA into Agrobacteria are known, including electroporation, freeze/thaw methods, and triparental mating. In some embodiments, a pMON316-based vector is used in the leaf disc transformation system of Horsch et al. Other commonly used transformation methods include, but are not limited to, microprojectile bombardment, biolistic transformation, and protoplast transformation of naked DNA by calcium, polyethylene glycol (PEG) or electroporation (Paszkowski et al., 1984, EMBO J. 3:2727-2722; Potrykus et al., 1985, Mol. Gen. Genet. 199:169-177; Fromm et al., 1985, Proc. Nat. Acad. Sci. USA 82:5824-5828; Shimamoto et al., 1989, Nature, 338:274-276.

To transgenic plants may be used to generate subsequent generations (e.g., T1, T2, etc.) by selfing of primary or secondary transformants, or by sexual crossing of primary or secondary transformants with other plants (transformed or untransformed).

The described CRISPR systems can be used to genetically modify an endogenous PCR1 genomic locus or to introduce a heterologous UGT78D-B gene into a plant. The plant can be a plant having another trait of interest. Delivery of the CRISPR system leads to insertions, deletions, or substitutions near a target sequence.

In some embodiments, the described modified plants have a reduced ability to catalyze attachment of a galactosyl group to the 3 position of a kaempferol molecule to form kaempferol 3-O-galactoside. In some embodiments, the modified plants have reduced male fertility. Mail fertility in the modified plants can be reduced by at least 25%, at least 30%, at least 35%, at least 40%, at least 45%, at least 50%, at least 60%, at least 65%, at least 70%, at least 75%, at least 80%, at least 85%, at least 90%, or at least 95% relative to a similar plant that does not contain a modified UGT78D-B gene. In some embodiments, the modified plants have reduced pollen tube growth. Pollen tube growth in the modified plants can be reduced by at least 25%, at least 30%, at least 35%, at least 40%, at least 45%, at least 50%, at least 60%, at least 65%, at least 70%, at least 75%, at least 80%, at least 85%, at least 90%, or at least 95% relative to a similar plant that does not contain a modified UGT78D-B gene. In some embodiments, the modified plants have reduced seed setting. Seed setting in the modified plants can be reduced by at least 25%, at least 30%, at least 35%, at least 40%, at least 45%, at least 50%, at least 60%, at least 65%, at least 70%, at least 75%, at least 80%, at least 85%, at least 90%, or at least 95% relative to a similar plant that does not contain a modified UGT78D-B gene.

The transgenic plant line can also contain one or more genes for herbicide tolerance, increased yield, insect control, other fungal disease resistance, virus resistance, bacterial disease resistance, germination and/or seedling growth control, enhanced animal and/or human nutrition, improved processing traits, or improved flavor, among others.

### V. Detection of a Modified Gene

Modification of a plant to modify a UGT78D-B genomic locus using any of the described CRISPR constructs can be detected or confirmed by any means known in the art for detecting genetic modifications.

In some embodiments, a modification can be detected in a genomic DNA sample. Genomic DNA samples include, but are not limited to, genomic DNA isolated directly from a plant, cloned genomic DNA, or amplified genomic DNA.

Genetic analysis methods include, but are not limited to, polymerase chain reaction (PCR)-based detection methods (for example, TaqMan assays), microarray methods, mass spectrometry-based methods and/or nucleic acid sequencing methods, including whole genome sequencing. In some embodiments, the detection of genetic modification in a sample of DNA, RNA, or cDNA may be facilitated through the use of nucleic acid amplification methods. Such methods specifically increase the concentration of polynucleotides that span a target site, or include that site and sequences located either distal or proximal to it. Such amplified molecules can be readily detected by gel electrophoresis, fluorescence detection methods, or other means.

In some embodiments, modification of a UGT78D-B genomic locus is detected by hybridization to allele-specific oligonucleotide (ASO) probes. ASO probes are described in U.S. Pat. Nos. 5,468,613 and 5,217,863, each of which is incorporated herein by reference. Single or multiple nucleotide variations in nucleic acid sequence can be detected in nucleic acids by a process in which the sequence containing the nucleotide variation is amplified, spotted on a membrane and treated with a labeled allele-specific oligonucleotide probe.

In some embodiments, a modified UGT78D-B genomic locus or gene is detected by a probe ligation method. Probe ligation methods are described in U.S. Pat. No. 5,800,944 (incorporated herein by reference) where a sequence of interest is amplified and hybridized to probes followed by ligation to detect a labeled part of the probe.

In some embodiments, microarrays can be used for detection of a modified UGT78D-B genomic locus or gene. Typing of target sequences by microarray-based methods is described in U.S. Pat. Nos. 6,799,122, 6,913,879, and 6,996,476, each of which is incorporated herein by reference.

In some embodiments, a modified UGT78D-B genomic locus or gene can be directly identified or sequenced using nucleic acid sequencing technologies. Methods for nucleic acid sequencing are known in the art and include parallel bead arrays, sequencing by ligation, capillary electrophoresis, electronic microchips, “biochips,” microarrays, parallel microchips, and single-molecule arrays.

In some embodiments, the modified UGT78D-B genomic locus or gene in a plant may be detected through the use of a nucleotide probe.

A polynucleotide probe may be labeled or unlabeled. A wide variety of techniques are readily available in the art for labeling a nucleotide probe. Nucleotide labels include, but are not limited to, radiolabeling, fluorophores, haptens, antibodies, antigens, enzymes, enzyme substrates, enzyme cofactors, and enzyme inhibitors. A label may provide a detectable signal by itself (e.g., a radiolabel or fluorophore) or in conjunction with other agents.

A probe may be an exact copy of a marker to be detected. A probe may also be a nucleic acid molecule comprising, or consisting of, a nucleotide sequence which is substantially identical to an inserted nucleic acid sequence. The term “substantially identical” may refer to nucleotide sequences that are more than 85% identical. For example, a substantially identical nucleotide sequence may be 86%, 87%, 88%, 89%, 90%, 91%, 92%, 93%, 94%, 95%, 96%, 97%, 98%, or 99% identical to the reference sequence.

A probe may also be a nucleic acid molecule that is “specifically hybridizable” or “specifically complementary” to an exact copy of the marker to be detected (“DNA target”). “Specifically hybridizable” and “specifically complementary” are terms that indicate a sufficient degree of complementarity such that stable and specific binding occurs between the nucleic acid molecule and the DNA target. A nucleic acid molecule need not be 100% complementary to its target sequence to be specifically hybridizable. A nucleic acid molecule is specifically hybridizable when there is a sufficient degree of complementarity to avoid non-specific binding of the nucleic acid to non-target sequences under conditions where specific binding is desired. Thus, an oligonucleotide probe is “specifically hybridizable” to a marker allele if stable and specific binding occurs between the oligonucleotide probe and the marker allele (e.g., a SNP marker) under stringent hybridization conditions, but stable and specific binding does not occur between the oligonucleotide probe and the wild-type allele at the marker position.

In some embodiments, a probe comprises a pair of primers designed to produce an amplification product, wherein the amplification product is directly or indirectly determinative for the presence or absence of an AFR and/or ARN resistance marker.

### VI. Marker-Assisted Selection

Described are markers and methods for marker-assisted selection (MAS) of a UGT78D-B genomic locus or gene. Use of MAS in breeding programs can be used to facilitate modification of UGT78D-B in diverse genetic backgrounds.

UGT78D-B gene markers useful for genotyping (mapping, tracking, identifying, analyzing) a UGT78D-B genomic locus in a plant are described. In some embodiments, a UGT78D-B gene marker comprises a detectable genetic marker linked, closely linked, tightly linked, or extremely tightly to a UGT78D-B genomic locus.

In some embodiments, a UGT78D-B marker comprises a PCR amplification product, a single nucleotide polymorphism (SNP), a restriction fragment length polymorphism (RFLP), an amplified fragment length polymorphism (AFLP), a simple sequence repeat (SSR), a simple sequence length polymorphism (SSLP), an insertion/deletion polymorphism (indel), a variable number tandem repeat (VNTRs), or a random amplified polymorphic DNA (RAPD) linked, closely linked, tightly linked, or extremely tightly linked to a UGT78D-B genomic locus.

With the described UGT78D-B gene markers, plants with modified male fertility can be rapidly and efficiently identified.

### VII. Brief Description of the Sequences

The nucleotide and amino acid sequences listed in the accompanying sequence listing are shown using standard letter abbreviations for nucleotide bases, and single-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 1
UGT78D-B orthologs

| | Coding | | Protein |
|---|---|---|---|
| | sequence | mRNA | Sequence |
| | SEQ ID NO: | SEQ ID NO: | SEQ ID NO: |
| Solanum lycopersicum | 1 | 2 | 3 |
| Nicotiana tabacum | 4 | | 5 |
| Capsicum annuum | 6 | | 7 |
| Solanum tuberosum | 8 | | 9 |

TABLE 2
UGT78D-B ortholog accession numbers

| | | Genomic | Protein |
|---|---|---|---|
| | mRNA | Sequence | Sequence |
| | (accession no.) | (accession no.) | (accession no.) |
| Solanum lycopersicum | | Solyc07g006720 | |
| Nicotiana tabacum | NW_015838499.1 | XM_016651521.1 | XP_016507007 |
| Capsicum annuum | XM_016723788.2 | NC_061117 | XP_016579274 |
| Solanum tuberosum | | | XP_006343538 |

TABLE 3
Exemplary UGT78D-B CRISPR guide RNA sequences

| | | SEQ |
|---|---|---|
| plant | guide RNA sequence | ID NO: |
| Solanum | ccaaagggtcacgtgctcgg | 10 |
| lycopersicum | | |
| Nicotiana tabacum | gtacccgagggatacgcgat | 11 |
| | ttctaaaatgcttcacaaaa | 12 |
| Capsicum annuum | ggtggactcgaagatattat | 13 |
| | tttccatttgcaacacatgc | 14 |
| Solanum tuberosum | ggtggtctcgaagagattat | 15 |
| | atgggaaaaaccataggaaa | 16 |

TABLE 4
UGT78D-B Sequences

Solanum lycopersicum UGT78D-B open reading frame (ORF) SEQ ID NO: 1
atgtctaattttcatgttgctgttatagcatttccttttgcaacacatgctgggcttttatatggactt
gttcaaaggctagcaagtacattaccaaatgtgacattcacattttttagcacaaaaaattcgaattct
tcgattttcaattcacaacattgttatagcaatacaaatgttaagccctacgacgtctctgatggcgtc
ccaaagggtcacgtgctcggaggtggtctcgaggagattataggattgttcttcaaatccgcgaagcaa
aatattgataatgctatgatttctgctgagaatgactcagggaaaaaaattacatgtgttattgctgat
gcatttatgtggtttactagtgatatggctcaacagtgtaatgttcattggattcctgtatggacttct
gcagctggatcactatctgttcatgttgatactgatcttattagagaaaatgttgggatacaaggaatt
gatggacgtgaagatgaagttttgaattttattcctggaatttctgaagtaagacttggtgatttgcct
ggtggagttgtttctggtgacctaaaatcgccattttcgatgatgcttcataaaatgggaaaaaccata
ggaaaaggaaaagccatatcaataaattcatttgaagagctagatcctcaaatggttcaatatctcaag
tctaaattcaacaacattcttaatgttggtccttttaacttaacttcaccaccacctttagcaaatgtt
gtagacaaatatgattgtattgagtggttaaatcatcaagagccaaactctgtggcatatattggattt
ggtactgttgcaacaccaccacctaatgagttagaagcaatggctcaagcacttgaagacaaaaaaatt
ccatttctttggtcaattaaagagaattttatgtcacattttccaaaaggattcttggaaaagacaagt
gagtatggtaaagttgtaccatgggtaccacaagtacaagttcttgaacatagtgccgttggagttttt
ataaatcactctggatggaattcagtactcgagagtatagcgtctggggtacctataatttgtaggcct
tttttcggtgatcatcatttgaatgcttggatggttgaaaaagtttggaaaattggagtgaaaattgaa
ggtggggttttcactaaaagtggtacaattgatgcacttgaatttgttttgtcaaaaaataaagaggaa
ttgaaggaacaaattgggatgtataaagaacttgctttaaaggctgttggaacaagtgggagttcaact
caaaatttcaacaagttggttgagattatcacttcttgttag
Solanum lycopersicum UGT78D-B mRNA SEQ ID NO: 2
tttactctctccatattaatttttattctattatctccataaactcctctaagaaattaaaaaaaaaaa
cacaaaaaaatgtctaattttcatgttgctgttatagcatttccttttgcaacacatgctgggctttta
tatggacttgttcaaaggctagcaagtacattaccaaatgtgacattcacattttttagcacaaaaaat
tcgaattcttcgattttcaattcacaacattgttatagcaatacaaatgttaagccctacgacgtctct
gatggcgtcccaaagggtcacgtgctcggaggtggtctcgaggagattataggattgttcttcaaatcc
gcgaagcaaaatattgataatgctatgatttctgctgagaatgactcagggaaaaaaattacatgtgtt
attgctgatgcatttatgtggtttactagtgatatggctcaacagtgtaatgttcattggattcctgta
tggacttctgcagctggatcactatctgttcatgttgatactgatcttattagagaaaatgttgggata
caaggtatacatatatattattcgtcgaaatgacagaatccataaagattattcaatagttgaacatca
ttgtgtttttctatccattatgaatgaaatgggaaaaatgacagatatacccctgaaatatcgtaaatt
tatatgcatatatatatatatatatatatatatatatatatatatattctctgttatacttttggaaca
ttgatgcgtctgttatacaaaagttagagcatatatgcccttcactctgacgaaagactgattatggat
acgtggcgtaatcttatccgtcgatccgatatttaataaaggtatagtacatagatatgccctttaact
ttgcctcatttcacatttataccctccaaatttgggtgttcacaagtagacacttaaacttgtatgaac
aaatagacacaaacgtcctgtatgacatcctacatgaaaatttgtgtcatacgtgtacaagtttgagtg
cctatttgtgcacactcaaagttggagggcacaaatgtaaaatgaggtaaagttaaagggtatatttat
gtattatacctttaataaatgtcgggtcggtagataagattatgacacatgtatgtccgttagtttgaa
gggtatatatactttatttttttatggtaagagcaccaatgtctcaaaagtatgacataaggtatttgc
atcaaagacaccaacaatgtcccaaaagtataacagggtatctacataccatttttcaatagtttgagg
gtacatttgtcctttattcccctgagtatgaaattgactgatattatgacgataataggtaggatagat
atcactataggaaaaagttgatttcaaaatgataatcaatacaatataaataaagatctagtatatgaa
gatatgttgatatttctgaagtacgatctaacttttcttttgatttattacatgttgtattagcgcgat
acgataaaaatcattcatttattttttgttatttatagagaactttgtcatgttcatgatgactaaaca
ttggatgaatgattaagactatagtatatgaagatatgttgatgtttctgaagtacgatctaatttttc
ttttgatttattacatattgtattagcgcgatacaataaaaatcattcatttttttttgtttgatagga
attgatggacgtgaagatgaagttttgaattttattcctggaatttctgaagtaagacttggtgatttg
cctggtggagttgtttctggtgacctaaaatcgccattttcgatgatgcttcataaaatgggaaaaacc
ataggaaaaggaaaagccatatcaataaattcatttgaagagctagatcctcaaatggttcaatatctc
aagtctaaattcaacaacattcttaatgttggtccttttaacttaacttcaccaccacctttagcaaat
gttgtagacaaatatgattgtattgagtggttaaatcatcaagagccaaactctgtggcatatattgga
tttggtactgttgcaacaccaccacctaatgagttagaagcaatggctcaagcacttgaagacaaaaaa
attccatttctttggtcaattaaagagaattttatgtcacattttccaaaaggattcttggaaaagaca
agtgagtatggtaaagttgtaccatgggtaccacaagtacaagttcttgaacatagtgccgttggagtt
tttataaatcactctggatggaattcagtactcgagagtatagcgtctggggtacctataatttgtagg
ccttttttcggtgatcatcatttgaatgcttggatggttgaaaaagtttggaaaattggagtgaaaatt
gaaggtggggttttcactaaaagtggtacaattgatgcacttgaatttgttttgtcaaaaaataaagag
gaattgaaggaacaaattgggatgtataaagaacttgctttaaaggctgttggaacaagtgggagttca
actcaaaatttcaacaagttggttgagattatcacttcttgttagtgaaactataatatattcttgaaa
aggggttttaatgattaattattaattccacttttttttttttttaaaaaaatatatatatatatatgt
atcaattgttatcatgaattgatcatcaatatttgtccatgtaacattgtttttgggaaaacatttgta
atgaaattgaaactcatgactttctagtcacacaacaaactcttaccattgcatcaatataatatactc
tctctcgttaa
Solanum lycopersicum UGT78D-B protein SEQ ID NO: 3
MSNFHVAVIAFPFATHAGLLYGLVQRLASTLPNVTFTFFSTKNSNSSIFNSQHCYSNTNVKPYDVSDGV
PKGHVLGGGLEEIIGLFFKSAKQNIDNAMISAENDSGKKITCVIADAFMWFTSDMAQQCNVHWIPVWTS
AAGSLSVHVDTDLIRENVGIQGIDGREDEVLNFIPGISEVRLGDLPGGVVSGDLKSPFSMMLHKMGKTI
GKGKAISINSFEELDPQMVQYLKSKFNNILNVGPFNLTSPPPLANVVDKYDCIEWLNHQEPNSVAYIGF
GTVATPPPNELEAMAQALEDKKIPFLWSIKENFMSHFPKGFLEKTSEYGKVVPWVPQVQVLEHSAVGVF
INHSGWNSVLESIASGVPIICRPFFGDHHLNAWMVEKVWKIGVKIEGGVFTKSGTIDALEFVLSKNKEE
LKEQIGMYKELALKAVGTSGSSTQNFNKLVEIITSC
Nicotiana tabacum UGT78D-B ORF SEQ ID NO: 4
atgtccaattatcatgttgctgttctagcattcccttttgcaacacatgctggccttttacatggactt
gtccaaaggctagcaaatacattacctaatgtaacattcacatttttcaacacctccaaatcaaattct
ttaattttctccaatcaaaccgacccacattgtactaatcttaagcccttcgatatctccgatggcgta
cccgagggatacgcgatcggagaaggaggaatcgaggagcttatagggttattttttaaatccgcgaaa
aataattttcagaatgctatatcagctgctgaggatgaaacgggaaaaaaaattacgtgcgttttcgcg
gatgcgtttatatggttttccggtgagattgcggaggaattaggcgttgattggattcctgtttggact
tctgcggcaggatcactgtctgttcatatttacactgattttattagagaaaatgttggggttcaagga
attgctggacgtgaagatgaaatgttaacattcattcctggattttctgaattaaggcttggtgatttg
cctagtggagttatatttggagatttaaaatcaccattttctaaaatgcttcacaaaatgggcaaaacc
ataacaaaagccaatgccttaccaataaattcctttgaagaattagaccctccaattgttgaaaatctc
aagtccaaattcaataattttcttaatgttggtccttttaacttaacctccccaccaccctcagcaaat
attatagacaaaaatggttgtattgaatggctagataatcaagaaccaaattctgtggcttatattgga
tttggcacagttgcaacaccaccacctaatgagttaaaatctatggctgaagcacttgaagaaagtaag
acaccttttctttggtcaattaaagatgattttatatcacattttccaaaagaatttttggaaaataca
agtgagtatggtaaaattgtaacatgggccccacaagtacaagttcttgaacatagttcaattggggtt
tttataaatcatagtggatggaattctgtgttggaaagtatagcttctggtgtgccaataatttgtagg
ccattttttggagaccatcatttgaattcttggatggttgaaaaagtgtggaaaattggagtgaaaatt
gaaggtggggtttttactaaaagtggtacaatggttgcacttgacttggttttatcaaaaaatagagag
aaattgaagcaacaaattggtatatataaagatcttgctctcaaggctgttggaccaagtgggagttca
actgaaaatttcaagaaattggttgagattatcacctctagcaattga
Nicotiana tabacum UGT78D-B protein SEQ ID NO: 5
MSNYHVAVLAFPFATHAGLLHGLVQRLANTLPNVTFTFENTSKSNSLIFSNQTDPHCTNLKPFDISDGV
PEGYAIGEGGIEELIGLFFKSAKNNFQNAISAAEDETGKKITCVFADAFIWFSGEIAEELGVDWIPVWT
SAAGSLSVHIYTDFIRENVGVQGIAGREDEMLTFIPGESELRLGDLPSGVIFGDLKSPFSKMLHKMGKT
ITKANALPINSFEELDPPIVENLKSKFNNELNVGPFNLTSPPPSANIIDKNGCIEWLDNQEPNSVAYIG
FGTVATPPPNELKSMAEALEESKTPFLWSIKDDFISHFPKEFLENTSEYGKIVTWAPQVQVLEHSSIGV
FINHSGWNSVLESIASGVPIICRPFFGDHHLNSWMVEKVWKIGVKIEGGVFTKSGTMVALDLVLSKNRE
KLKQQIGIYKDLALKAVGPSGSSTFNFKKLVEIITSSN
Capsicum annuum UGT78D-B ORF SEQ ID NO: 6
atgtctaataatttacatgttgctgttcttgcatttccatttgcaacacatgctggtcttttactagga
cttatacaaaggctatcaaattcattaccaaatgttacatttacatttttcaatactaaaagatcaaat
tcttcaatatttacacaagataataatacaaatattaagccatttgatatccctgatgggaaacctgag
agtcatgtacatggaggtggagatgaaggtggactcgaagatattataggtttattcttcaaatccgcg
aaaaataatgtgacaaatgccatcgcgactgctgagaatgactcagggaagaagattacgtgtgtcata
gctgacgcgtttatgtggttttcgagtgagatcaccgaggaactaggaattagttggattccagtatgg
acttccgcggcaggatcactctctgtgcatgttcataccgatcttattagagaaaacgttgggacccaa
ggaattgatggacgcgaagatgaaattttgaaatttattccgggttttttcgaagtaagacttggcgat
ttacctggaggagttgtcgtaggagatctgaaaacgtcattttctatgatgcttcacaaaatgggccaa
acaataggtaaaggaacagccataccaataaattcatttgaagaattggaccctccaattgtccaaaat
ctgaaatccaaattcaaaaattttctcaatattggaccatttaatttaacttcaccaacaccattagca
aatgtggttgacaaagatggttgtattgaatggttagacaaacaaaaaactaattctgtggcatatatt
ggatttggcactgtagcaacaccaccacctaatgagttaaaagcaatggctgaagcacttgaagagaaa
aaaataccatttctatggtcaattaaagagaaatttattgcaaattttccaaatggattcttggaaaaa
acaaaaatgtttggtaaagttgtgtcatgggcaccacaagtacaagttcttgaacataaagcgatcggc
gtttttataaatcactcaggatggaattcggttctggagagcatagcttctggcgtgcctataatatgt
aggccgtttttcggagatcatcatttgaattcttggatggttgagaaagtttggaaaattggtgtgaaa
attgaaggtggggtttttactaaaagtggtacaattgttgcacttgatttggttttgtctaaaaataaa
gaagaatttaagaaacaaattggaatgtacaaagaattagttcacaatgctgttggaccaaatggaagt
tcaacgcaaaatttcaagaaattggttgagattatcacctcttgttag
Capsicum annuum UGT78D-B Protein SEQ ID NO: 7
MSNNLHVAVLAFPFATHAGLLLGLIQRLSNSLPNVTFTFENTKRSNSSIFTQDNNTNIKPFDIPDGKPE
SHVHGGGDEGGLEDIIGLFFKSAKNNVTNAIATAENDSGKKITCVIADAFMWFSSEITEELGISWIPVW
TSAAGSLSVHVHTDLIRENVGTQGIDGREDEILKFIPGFFEVRLGDLPGGVVVGDLKTSFSMMLHKMGQ
TIGKGTAIPINSFEELDPPIVQNLKSKFKNELNIGPFNLTSPTPLANVVDKDGCIEWLDKQKTNSVAYI
GFGTVATPPPNELKAMAEALEEKKIPFLWSIKEKFIANFPNGFLEKTKMFGKVVSWAPQVQVLEHKAIG
VFINHSGWNSVLESIASGVPIICRPFFGDHHLNSWMVEKVWKIGVKIEGGVFTKSGTIVALDLVLSKNK
EEFKKQIGMYKELVHNAVGPNGSSTQNFKKLVEIITSC
Solanum tuberosum UGT78D-B ORF SEQ ID NO: 8
atgtctaattatcatgttgctgttatagcatttccttttgcaacacatgctggccttttatatggactt
gttcaaaggctagcaaatacattaccaaatgtgacattcacattttttagcacaaaaatttcgaattct
tcaatttttaattcacaaaattataattatagaaatacaaatcttaagccctatgacgtctctgatggc
gtcccagagggtcacgtgctcggaggtggtctcgaagagattataggattattcttcaaatccgcgaag
aaaaatattcataatgctataatttctgctgagaatgactcagggaaaaaaattacatgtgttattgcg
gacgcgtttatgtggttttcgagtaatatggctgaagagtttaacgttagttggattcctgtatggact
tccgcagctggatcactctctgttcatgttgataccgatgttataagacaaaatgttgggatccaagga
attgatggacgcgaagatgaaattttgaaatttattcctggaatttctgaagtaagacttggtaatttg
cctggtggaattgtttctggagacctaaaatcgccattttcgatgatgcttcataaaatgggaaaaacc
ataggaaaaggaaaagccataccaataaattcatttgaagaattagaccctcaaatggttcaatatctc
aagtctaaattcaacaatattcttaatgttggtccttttaatttaacttcaccaccacctctagcaaat
gttgtagacaaatatgattgtattgagtggttaaatagtcaagaaccaaattctgtggcatatattgga
tttggtactgttgcaacaccaccacctaatgagttaaaagctatggctcaagcacttgaagaaaaaaaa
attccatttatttggtcaattaaagagaattttatgtcacattttccacaagggttcttggaaaacaca
agtgagtatggtaaagttgtaccatgggtaccacaagtacaagttcttgaacatagtgcgattggagtt
tttataaatcactctggatggaattcggttctggagagcatagcttctggggtgcctataatttgtagg
cctttttttggagatcatcatttgaatgcttggatggttgagaaagtttggaaaattggtgtgaaaatt
gaaggtggggtttttacaaaaagtggtacaattgatgcacttgacttggttttgtcaaaaaatagagtg
gaattgaaggaacaaattggaatgtacaaagaacttgctttaaaggctgttggaacatgtgggagttca
actcaaaatttcaacaagttggttgagattatcacttcttgttaa
Solanum tuberosum UGT78D-B Protein SEQ ID NO: 9
MSNYHVAVIAFPFATHAGLLYGLVQRLANTLPNVTFTFFSTKISNSSIENSQNYNYRNTNLKPYDVSDG
VPEGHVLGGGLEEIIGLFFKSAKKNIHNAIISAENDSGKKITCVIADAFMWFSSNMAEEFNVSWIPVWT
SAAGSLSVHVDTDVIRQNVGIQGIDGREDEILKFIPGISEVRLGNLPGGIVSGDLKSPFSMMLHKMGKT
IGKGKAIPINSFEELDPQMVQYLKSKFNNILNVGPFNLTSPPPLANVVDKYDCIEWLNSQEPNSVAYIG
FGTVATPPPNELKAMAQALEEKKIPFIWSIKENFMSHFPQGFLENTSEYGKVVPWVPQVQVLEHSAIGV
FINHSGWNSVLESIASGVPIICRPFFGDHHLNAWMVEKVWKIGVKIEGGVFTKSGTIDALDLVLSKNRV
ELKEQIGMYKELALKAVGTCGSSTQNFNKLVEIITSC

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### EXAMPLES

### Example 1. Organ-Specific Flavonol Profiles in Tomato

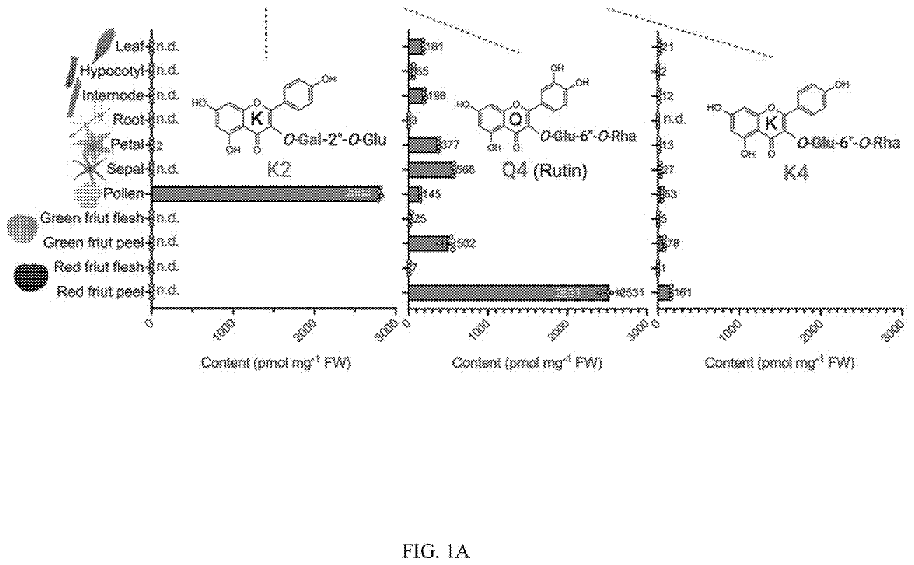

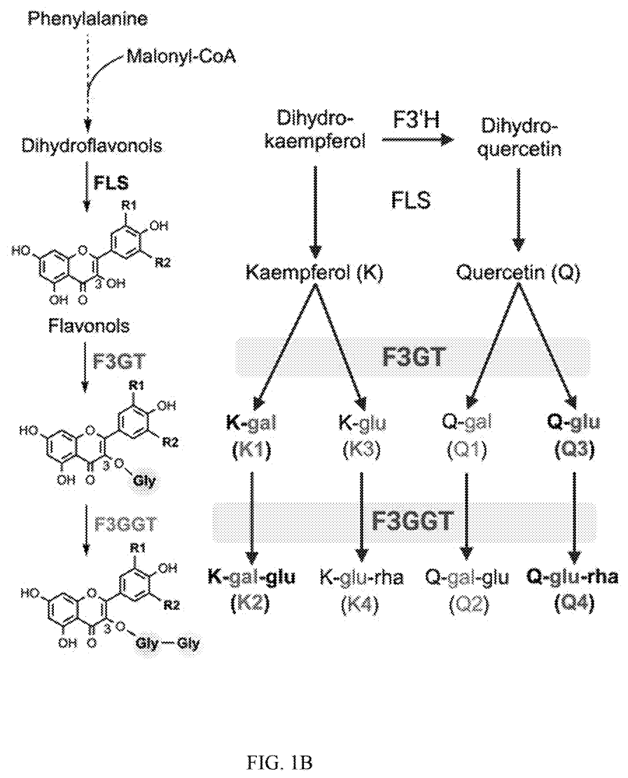

We analyzed methanol soluble metabolites from eleven different organs or tissues of the tomato plant using High-Performance Liquid Chromatography (HPLC). The analyzed organs included mature pollen grains, leaves, hypocotyls, internodes, roots, petals, sepals, as well as the fruit flesh and peel of both green and red fruits. In all organs except pollen grains, two flavonol rutinosides, quercetin 3-O-rhamnosyl (1→6) glucoside (Q-glu-rha, also known as rutin, Q4) and kaempferol 3-O-rhamnosyl (1→6) glucoside (K-glu-rha, K4), were predominantly accumulated, with Q4 (rutin) being the most abundant flavonol. Pollen predominantly accumulated a compound that appear in “Peak 1.” This peak was below the level of detection in all other tested organs except petal. To determine the structure of this pollen specific compound, we collected the HPLC eluent corresponding to the retention time of Peak 1 for MS analysis. The content of eluent exhibited an m/z value of 611.1594 in positive mode, which corresponding to a calculated exact mass of 610.1521 Da. Further fragmentation analysis of the precursor ion revealed two key fragment ions with m/z values of 287.0540 and 465.1009, corresponding to kaempferol and kaempferol-monohexose, respectively. The m/z differences between the parental ion and two fragments (324.1054 and 146.0512) suggested that Peak 1 represented a kaempferol di-glycoside comprising two hexose units. It was previously shown that the most abundant flavonol glycoside in pollen grains of petunia, a member of the Solanaceae family, is kaempferol 3-O-glucosyl (1→2) galactoside (K-gal-glu). We therefore analyzed the methanolic extract of petunia pollen grains and found that the retention time and UV absorbance spectra of Peak 1 exactly matched that of K-gal-glu. Thus, we concluded that Peak 1 is kaempferol 3-O-glucosyl (1→2) galactoside (K-gal-glu, hereafter called K2), which was exclusively accumulated in pollen at 2,804 μmol mg-1 FW (FIG. 1A).

### Example 2. Identification of the Enzymes Responsible to Produce Organ-Specific Flavonol Glycoside

Kaempferol (K) containing either glucose or galactose at the 3-OH position requires flavonol 3-O-glycosyltransferase (F3GT) activity (FIG. 1B). The production of flavonol 3-O-diglycosides (e.g., K-gal-glu (K2)), requires flavonol 3-O-glycoside: glycosyltransferase (F3GGT) activity (FIG. 1B). To identify the UGTs with F3GT or F3GGT activities, we conducted a phylogenetic analysis using protein sequences of 182 putative UGTs identified from the tomato genome (SL4.0 build; ITAG4.0 annotation). Our analysis also included 13 previously characterized F3GTs and F3GGTs from Arabidopsis, petunia, eggplant, tea, grape, barley, and maize. Two UGT homologs, Solyc10g083440 and Solyc07g006720, were clustered with other characterized F3GTs in the F3GT clade. It was previously shown that Solyc10g083440 (S/UGT78D-A) converts quercetin to quercetin 3-O-glucoside. The function of Solyc07g006720 (hereafter called S/UGT78D-B) was previously unknown. According to the public database, SIUGT78D-B is highly expressed in pollen, which we confirmed experimentally.

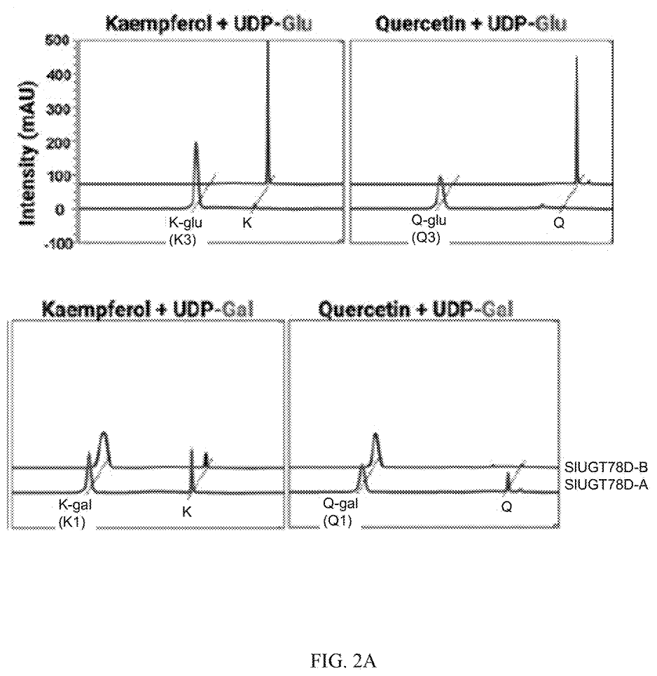

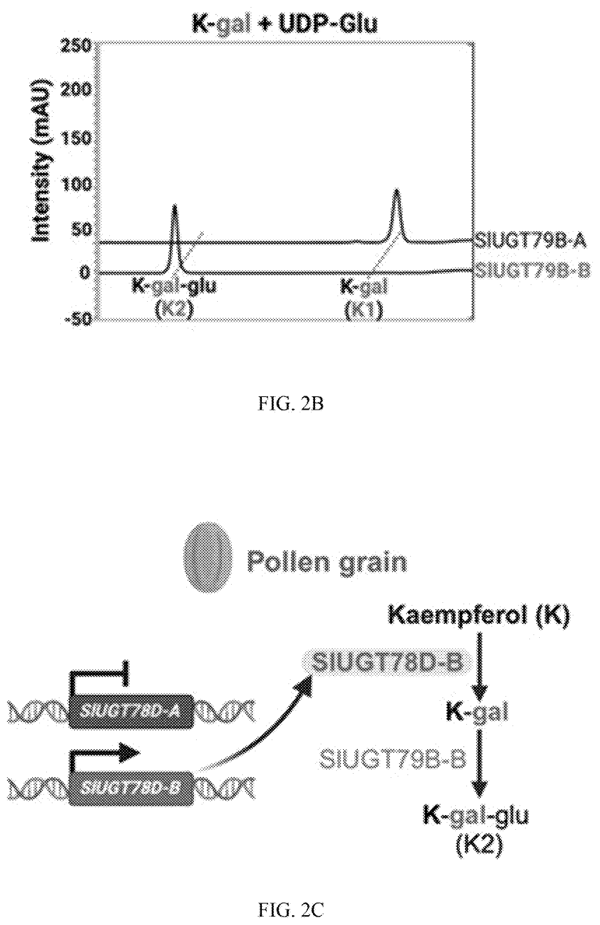

Then, we conducted enzyme activity tests using recombinant S/UGT78D-A and SZUGT78D-B with two sugar donors, UDP-glucose and UDP-galactose, and two sugar acceptors, kaempferol and quercetin. S/UGT78D-A produced flavonol 3-O-glucosides (K3 and Q3) or flavonol 3-O-galactosides (K1 and Q1) (FIG. 2A), indicating that it has both flavonol 3-O-glucosyltrasferase (F3Glu) and 3-O-galactosyltransferase (F3Gal) activities. In contrast, SIUGT78D-B produced flavonol 3-O-galactosides, but not flavonol 3-O-glucosides, suggesting that S/UGT78D-B functions as a flavonol 3-O-galactosyltransferase (F3Gal) but lacks 3-O-glucosyltransferase activity (F3Glu) (FIG. 2A).

The production of K-gal-glu (K2) requires additional UGT enzyme that can add glucose to K-gal at its 2″ position (2″-O-glucosyltransferase activity), which requires flavonol 3-O-glycoside: glycosyltransferase (F3GGT) activity (FIG. 1B). In the F3GGT clade, which includes characterized F3GGTs from Arabidopsis and petunia, five putative tomato UGTs were included. Solyc09g059170 (S/UGT79B-A) clustered closely with Petunia flavonoid 3-O-glucoside: 6″-O-rhamnosyltransferase, while Solyc10g008860 (S/UGT79B-B) clustered with petunia flavonol 3-O-glycoside: 2″-O-189 glucosyltransferase. The other three enzymes grouped with Arabidopsis F3GGTs. However, only SIUGT79B-B is expressed in pollen grains, while the other four candidates show no expression in pollen grains according to a public database. Thus, evidence indicates that S/UGT79B-B is the F3GGT responsible for K2 production in pollen grains. In support of this, we demonstrated that recombinant S/UGT79B-B showed kaempferol 3-O-glycoside: 2″-O-glucosyltransferase activity and produced K-gal-glu (K2) using K-gal and UDP-Glucose, while recombinant S/UGT79B-A did not show the same activity (FIG. 2B). Our results indicate that differentially expressed flavonol glycosyltransferases and their sugar specificities shape the distinctive organ-specific flavonol glycoside profiles in tomato. In pollen, the repression of SIUGT78D-A together with the activation of SIUGT78D-B enriches kaempferol galactosides (FIG. 2C).

### Example 3. Key Residues Determining Sugar Specificity of SIUGT78D-B

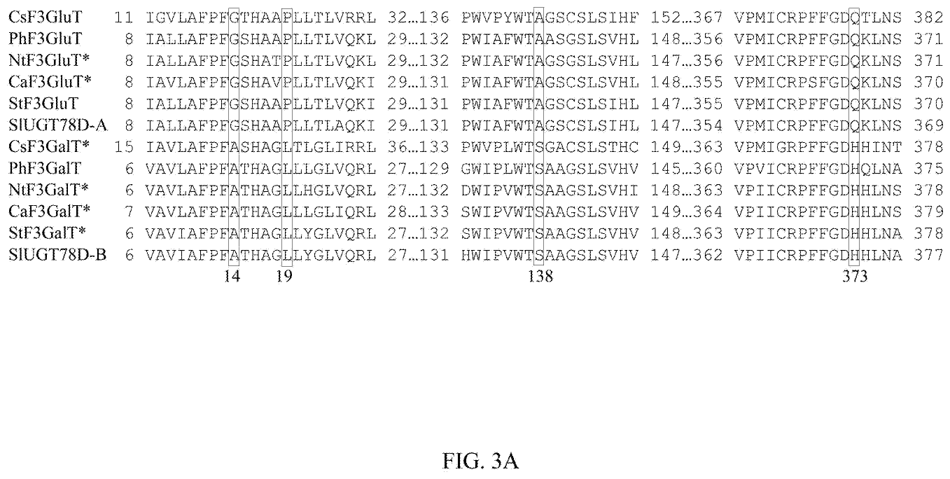

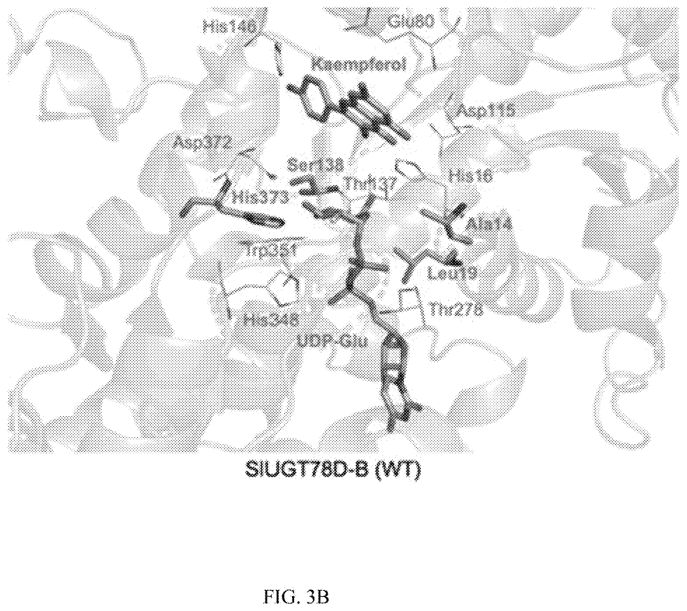

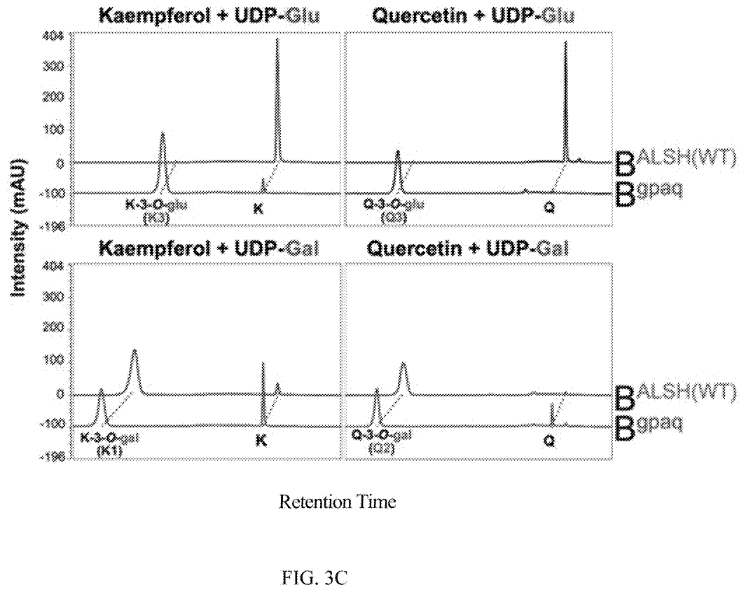

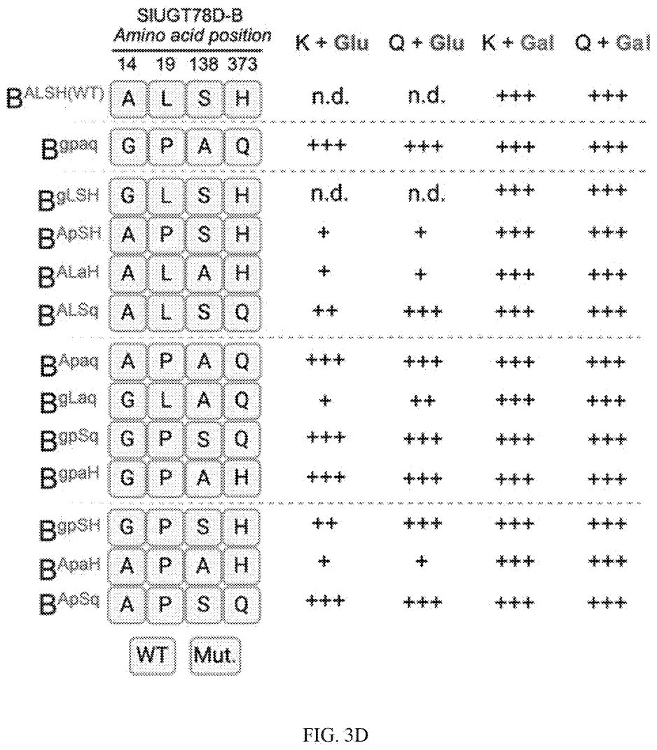

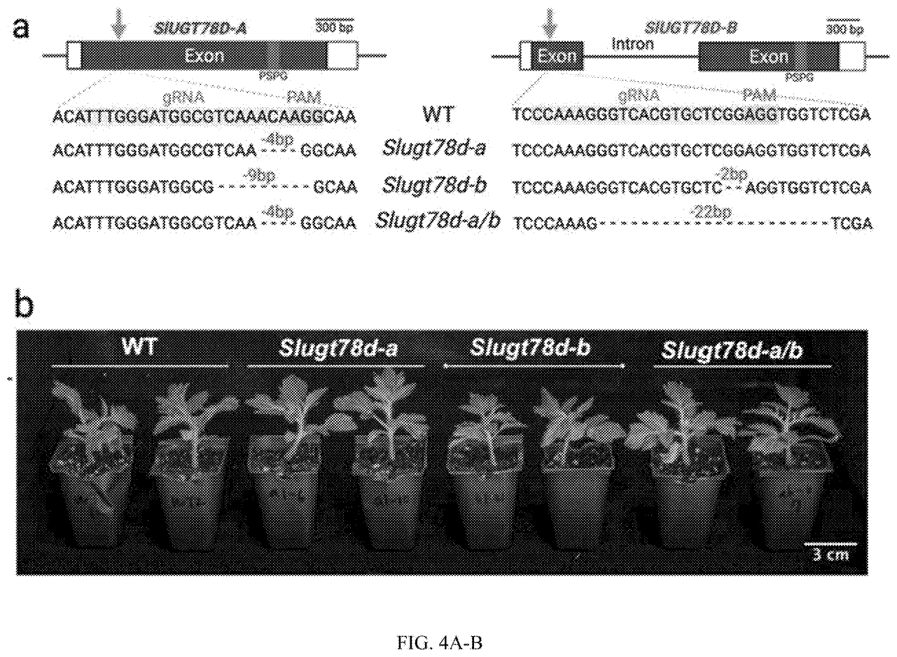

To determine the molecular basis of their differing sugar preferences, we compared the amino acid sequences of S/UGT78D-A and S/UGT78D-B homologs. Our analysis included the characterized CsUGT78A14 (flavonol 3-O-glucosyltransferase, F3GluT) and CsUGT78A15 (flavonol 3-O-galactosyltransferase, F3GalT) from Camellia sinensis47 and five Solanaceae species (identified via an NCBI GenBank BLAST search). Phylogenetic analysis grouped the homologs into two distinct clades: the F3GluT group (including S/UGT78D-A) and the F3GalT group (including S/UGT78D-B). The homologs within each group shared over 75% sequence similarity, while similarity between groups was less than 50%. We identified 11 amino acids conserved in each group, and also identified 17 residues predicted to be exposed in the ligand-binding pocket based on studies of GT1 from Vitis vinifera, leading us to pinpoint four residues in SZUGT78D-B (14A, 19L, 138S, and 373H) which position near the predicted sugar binding site (FIG. 3A-B).

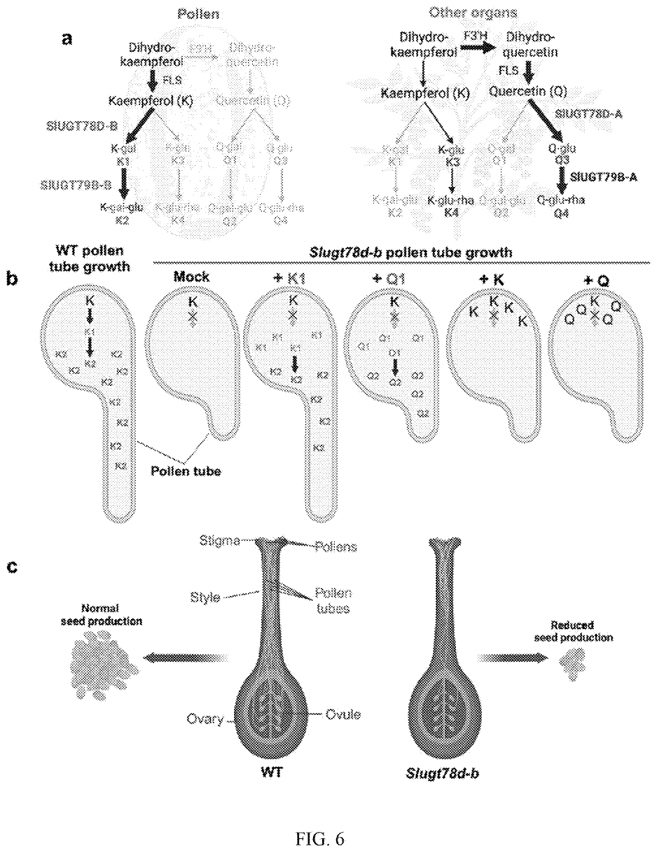

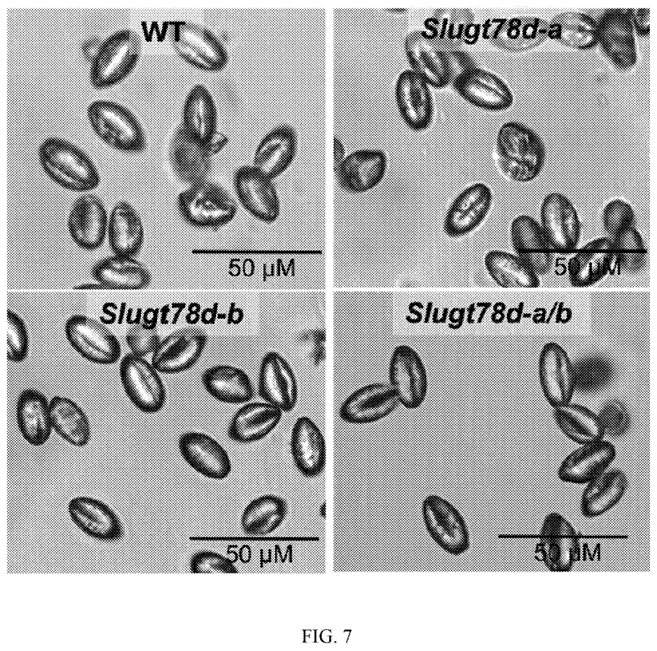

To test the significance of the identified four residues in sugar specificity, we generated various modified S/UGT78D-B proteins with substitutions at these positions with the corresponding ones from S/UGT78D-A. S/UGT78D-Bgpaq carrying all four amino acid substitutions (A14G, L19P, S138A, H373Q) gained flavonol 3-O-glucosyltransferase activity while retaining its galactosyltransferase activity, thereby resembling the bifunctional activity of SZUGT78D-A (FIG. 3C-D. FIG. 6). This suggests that these residues are essential for sugar specificity individually or in combination. To further narrow down key residues, we generated additional modified S/UGT78D-B with single, double, and triple amino acid substitutions and they all exhibited bifunctional activity, except for the single substitution at A14 (FIG. 3D and FIG. 6). Given that any single amino acid substitution of L19, S138, and H373 enables S/UGT78D-B acquire glucosyltransferase activity, there is a selective pressure to restrict its glucosyltransferase activity but maintaining their galactosyltransferase activity. Notably, introducing the corresponding residues from S/UGT78D-B into S/UGT78D-A abolished glucosyltransferase and galactosyltransferase activities, further confirming their critical role.

### Example 4. Disruption of SIUGT78D-B Leads to Impaired Pollen Tube Growth and Reduced Seed Yield

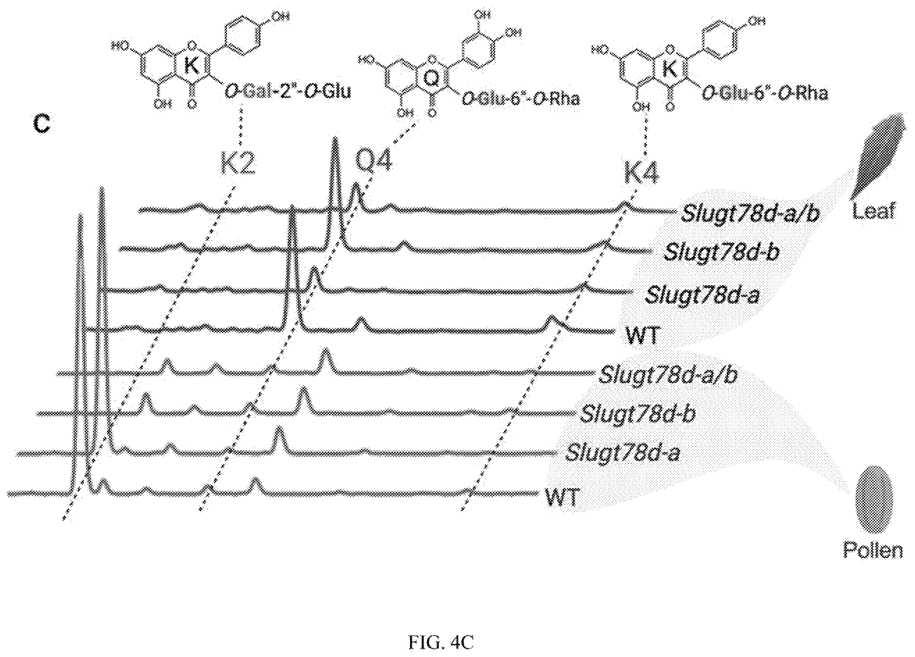

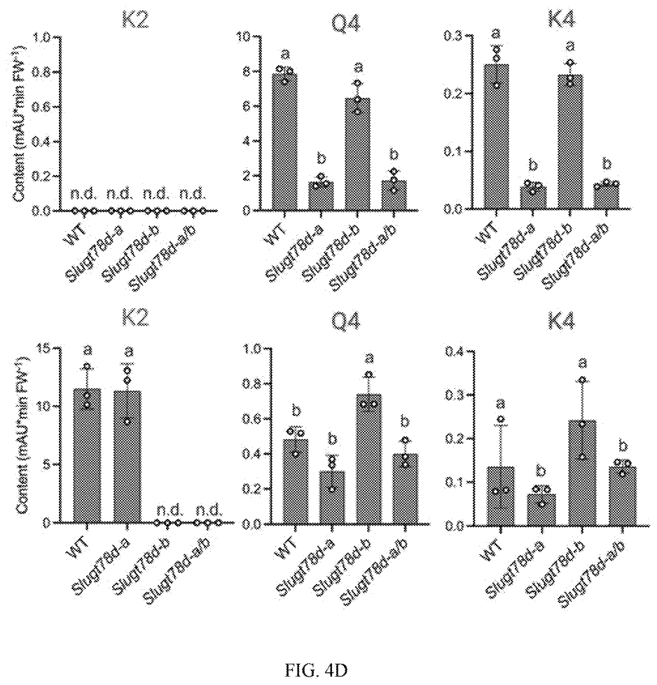

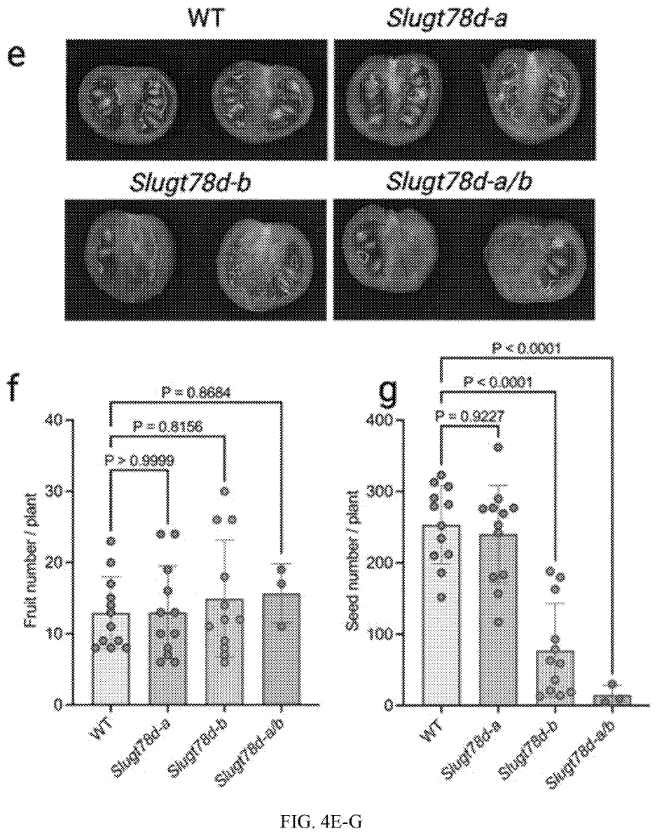

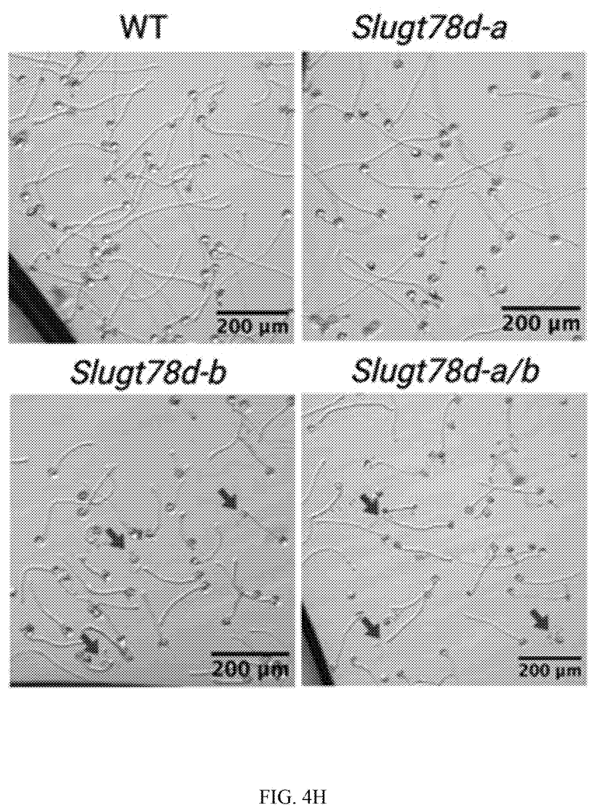

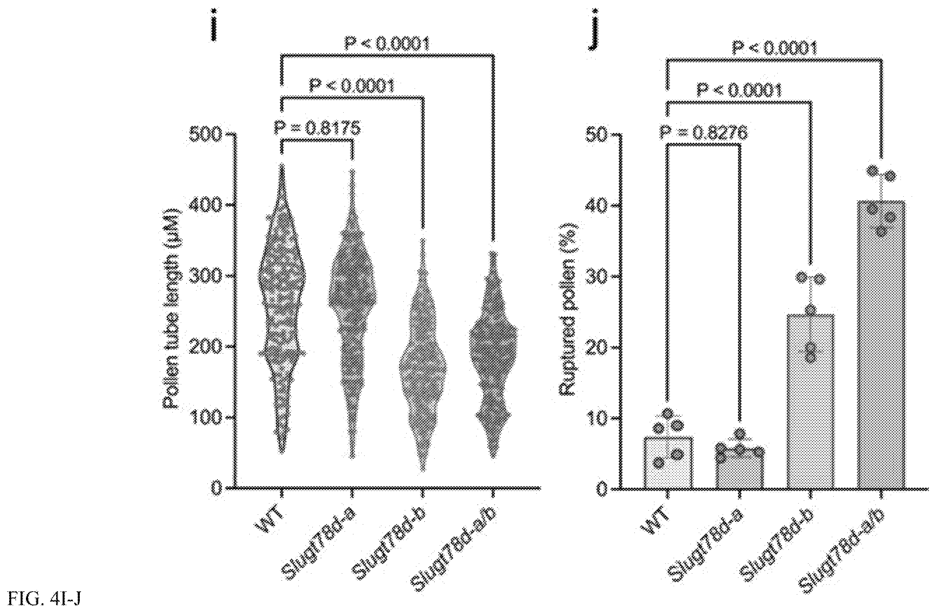

To determine any physiological functions of flavonol glycosides in tomato, we disrupted SZUGT78D-A and S/UGT78D-B using a CRISPR/Cas9 system that simultaneously targets both genes. Three independent homozygous mutant lines (named Slugt78d-a, Slugt78d-b, and Slugt78d-a/b) were established (FIG. 4A). The Slugt78d-a mutant has a 4 bp deletion mutation in the exon of SIUGT78D-A, resulting in an early stop codon, while SIUGT78D-B remained unaltered. The Slugt78d-b mutant has a 2 bp deletion in the first exon of SIUGT78D-B that causes an early stop codon. It also carries a 9 bp deletion in SIUGT78D-A that removes three amino acids. Finally, the Slugt78d-a/b mutant has a 4 bp deletion in the exon of SIUGT78D-A and a 22 bp deletion in the first exon of SIUGT78D-B, both resulting in early stop codons. All frameshift mutations resulted in truncated proteins lacking the Plant Secondary Product Glycosyltransferase (PSPG) motif, which is required for the glycosyltransferase reaction.

None of the mutants exhibited any visible changes in vegetative growth or development (FIG. 4B). However, both Slugt78d-a and Slugt78d-a/b mutants showed 80% reduction in the levels of flavonol glucosides, K-glu-rha (K4) and Q-glu-rha (Q4) in their leaves compared to wild type and Slugt78d-b mutant (FIG. 4C, FIG. 4D). In contrast, the levels of K4 and Q4 in the pollen of these mutants were not substantially changed compared to wild type (FIG. 4C, FIG. 4D). While the Slugt78d-b mutant carries 9 bp deletion in SIUGT78D-A gene, levels of K4 and Q4 remained unchanged, suggesting that the three amino acid deletion did not affect its glucosyltransferase activity (FIG. 4A, FIG. 4C, FIG. 4D). Thus, we consider Slugt78d-b to be a single null mutant of SIUGT78D-B.

The pollen-specific flavonol, K-gal-glu (K2), was not detected in either the Slugt78d-b or the Slugt78d-a/b pollen. In contract, the Slugt78d-a mutant accumulated wild-type levels of K2 (FIG. 4C, FIG. 4D) in its pollen. SIUGT78D-A expression was not induced in the Slugt78d-b pollen, suggesting that S/UGT78D-B is the sole enzyme responsible for the 3-O-galactosylation of kaempferol in pollen.

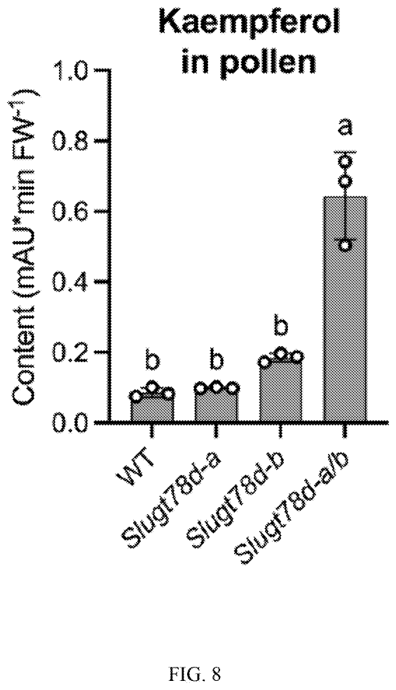

Fruit shape and the total number of fruits per plant remained unaltered in these mutants compared to wild type (FIG. 4E-F). However, the number of seeds per plant was significantly reduced in Slugt78d-b and Slugt78d-a/b compared to wild type and Slugt78d-a (FIG. 4G). Because successful seed production requires effective fertilization, and flavonoid deficient mutants in several species were reported to display defective pollen, we hypothesized that the reduced seed yield might be related to pollen development. Surprisingly, the overall shape and size of pollen grains were indistinguishable across all genotypes (FIG. 7). However, pollen tube length of Slugt78d-b and Slugt78d-a/b was significantly shorter than those from wild type and Slugt78d-a (FIG. 4H-I). The number of ruptured pollen tubes was also increased in Slugt78d-b and Slugt78d-a/b (FIG. 4H, FIG. 4J). Both Slugt78d-b and Slugt78d-a/b mutants lack the pollen-specific kaempferol 3-O-glucosyl (1→2) galactoside (K2) due to the absence of functional S/UGT78D-B. It was previously shown that the tomato flavonol deficient mutant showed deformed pollen grain shape and abnormal pollen tube growth, which accompanied with increased levels of reactive oxygen species (ROS) and the supplementation of kaempferol reduced the levels of ROS and rescued defective pollen (Muhlemann J K et al. “Flavonols control pollen tube growth and integrity by regulating ROS homeostasis during high-temperature stress.” Proc. Natl. Acad. Sci. 115, E11188-E11197 (2018) and Rutley N. et al. “Enhanced Reproductive Thermotolerance of the Tomato high pigment 2 Mutant Is Associated With Increased Accumulation of Flavonols in Pollen.” Front. Plant Sci. 12, (2021)). However, we found that steady state levels of free kaempferol in Slugt78d-b and Slugt78d-a/b mutants were not reduced compared to the wild type (FIG. 8). In fact, Slugt78d-a/b mutants accumulated seven folds higher level of free kaempferol compared to controls.

As kaempferol aglycone is not limited in Slugt78d-b pollen, we hypothesized that kaempferol 3-O-galactoside or its derivatives such as K2 is associated with proper pollen tube growth.

### Example 5. The Galactosylated Kaempferol, but Neither the Aglycones Nor Quercetin Galactosides, Rescued Defective Pollen Tube Growth

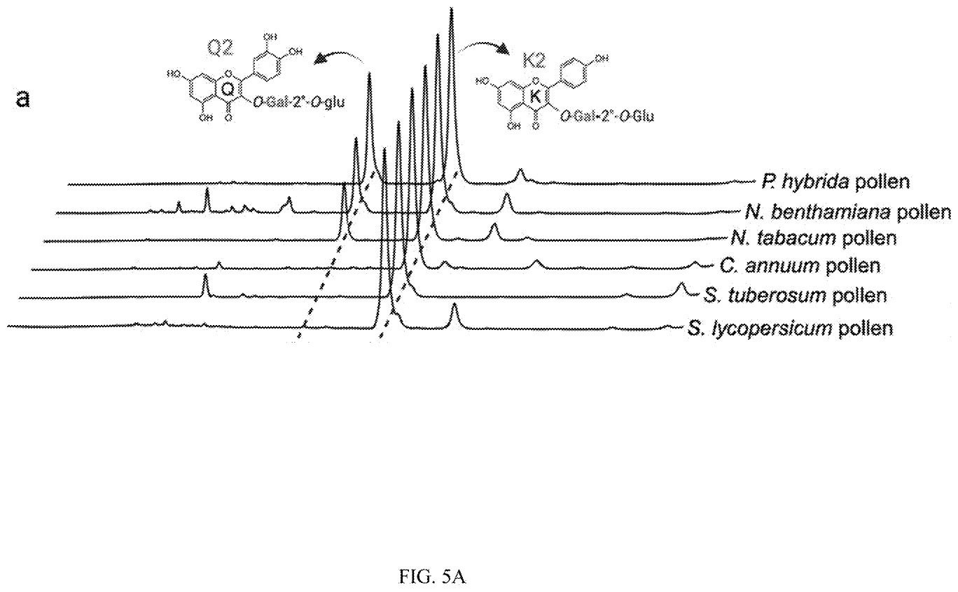

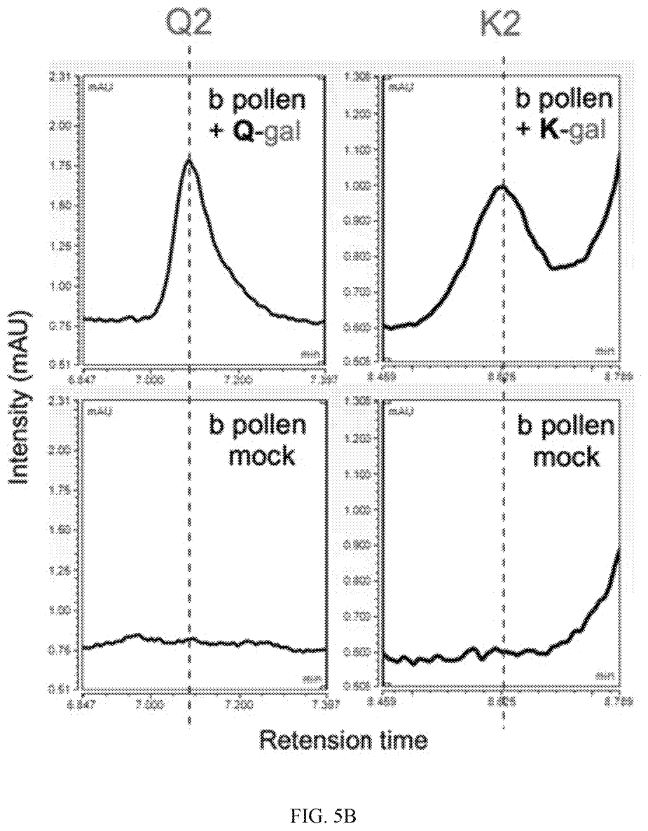

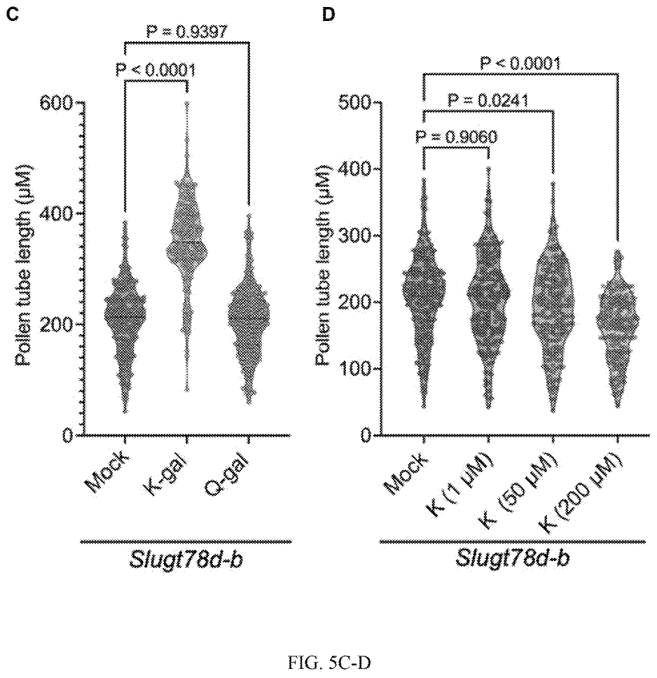

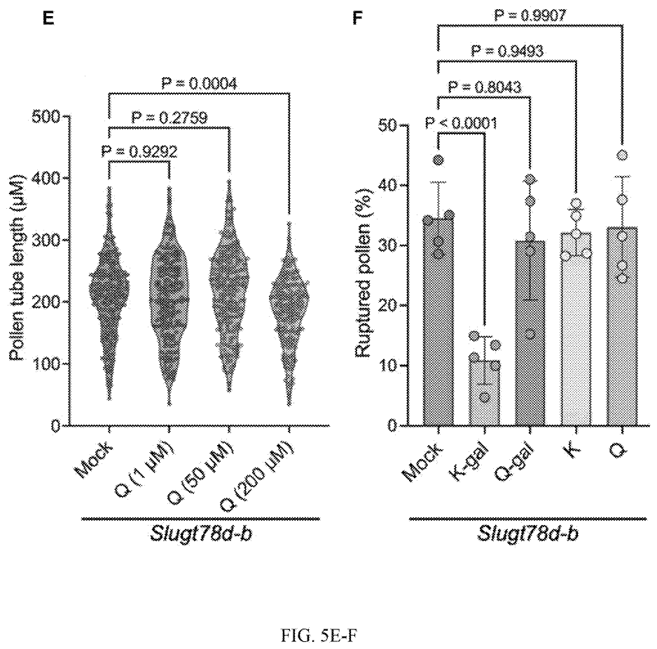

Since the pollen-specific galactoside, K-gal-glu (K2), was detected in the pollen of both tomato and petunia pollen and homologs of the K2 biosynthetic enzymes, S/UGT78D-B and SZUGT79B-B, are found in other solanaceae plants, we hypothesized that K2 might be conserved across other Solanaceae species. To test it, we analyzed methanolic extracts of pollen from Solanum tuberosum (potato), Capsicum annuum (chili pepper), and two tobacco species (Nicotiana tabacum and Nicotiana benthamiana) and found that K2 was the most abundant flavonol in all samples (FIG. 5A). Interestingly, Q-gal-glu (Q2) was detected in petunia and two tobacco species, but not in chili pepper, potato, and tomato (FIG. 5A). Considering that the chili pepper, potato, and tomato diverged more recently than tobacco and petunia, K2 appears to have been maintained during the evolution, whereas Q2 was eliminated.

To determine whether pollen-specific flavonols play any roles in pollen tube development, we conducted chemical complementation tests by supplying flavonols to germinating Slugt78d-b pollen. When K-gal and Q-gal were supplied to the Slugt78d-b pollen, K2 and Q2 were detected, whereas they were absent in the mock control (FIG. 5B). This suggests that both K-gal and Q-gal were successfully converted to K2 and Q2, respectively, in pollen. Notably, K-gal supplementation increased pollen tube length and reduced the number of ruptured pollen tubes of Slugt78d-b (FIG. 5C, FIG. 5F). However, the same concentration of Q-gal did not alleviate the defective pollen phenotypes (FIG. 5C, FIG. 5F). We also tested various concentrations of their aglycones, kaempferol and quercetin, but none of them improved the defective pollen phenotypes (FIG. 5D, FIG. 5F). This finding further confirmed that kaempferol aglycone was not a limiting factor for pollen tube growth, as its levels in Slugt78d-b pollen were seven times higher than in the wild type.

S/UGT78D-B having strict sugar specificity toward galactose is expressed almost exclusively in pollen. K2 was undetectable in the pollen of Slugt78d-b plants indicating SZUGT78D-B is the only kaempferol 3-O-galactosyltransferase in pollen and no other promiscuous UGTs can compensate for its activity in pollen.

We have demonstrated the necessity of pollen-specific kaempferol galactoside for proper pollen tube growth. Further the requirement for kaempferol galactoside that cannot be compensated for by other aglycones or quercetin galactosides. Exogenously applied K2 was able to rescue defective pollen tube growth.

Further investigations of the amino acid sequences of enzymes show that specific conserved amino acids in S/UGT78D-B are essential for restricting its activity to galactosylation. Among four conserved amino acids (Ala14, Leu19, Ser138, and His373) of S/UGT78D-B that are predicted to face the sugar binding pocket, Leu19, Ser138, and His373 were important is maintaining galactose specificity.

We demonstrated that specific glycosylation of kaempferol is involved in for proper pollen tube growth in tomato. Further, we demonstrated that modification of plants to decrease SIUGT78D-B expression resulted in decrease pollen tube growth and male infertility. Supplementation of Slugt78d-b plants with glycosylated kaempferol rescued defective pollen tube growth.

### Example 6. Materials and Methods

A. Plant material and growth conditions. The Micro-Tom variety was used as the wild-type plant and for generating CRISPR-Cas9-mediated mutant lines. All tomato plants were germinated in 3.7×3.7×5.8 cm seedling trays and then transplanted into 9×9×9 cm individual pots 3 weeks after germination, where they were grown until fruit and seed production. Tomato plants were grown at 22° C.±1° C. under a 16-hour day/8-hour night photoperiod.

B. Generation of CRISPR-Cas9-mediated mutant lines. To disrupt S/UGT78D-A and S/UGT78D-B using the CRISPR-Cas9 system, gRNAs targeting both genes were designed using the CHOPCHOP software. Specifically, two 20 bp gRNAs (5′-ATTTGGGATGGCGTCAAACA-3′ SEQ ID NO: 17) and (5′-CCAAAGGGTCACGTGCTCGG-3′ SEQ ID NO:10) were selected for SIUGT78D-A and SIUGT78D-B, respectively. Then, a DNA fragment containing a BsaI restriction site, the SIUGT78D-A gRNA, a gRNA scaffold, the U6-26t and U6-6P sequences, the SIUGT78D-B gRNA, and another BsaI restriction site was synthesized (Twist Biosciences, South San Francisco, CA, USA), which was cloned into the binary vector pHSE401 (Addgene, #62201) using the BsaI restriction site. The recombinant pHSE401 vector was then transformed into A. tumefaciens strain GV3101 and inoculated into cut cotyledons following a tomato transformation method. The mutant lines were selected according to the sequencing results of PCR products amplified with primers no. 1 and 2 (AGACCTCAAATCAAAGCTCCA (SEQ ID NO:18) and AGTCTTGACTCCGTTGTCTCT (SEQ ID NO:19)) for SIUGT78D-A, and no. 3 and 4 (AGCATTTCCTTTTGCAACACA (SEQ ID NO:20) and AGTGATCCAGCTGCAGAAGT (SEQ ID NO:21)) for SIUGT78D-B.

C. Phylogenetic analysis. Putative tomato UGTs were identified by querying the UniProt database using “UDP-glycosyltransferase” as the search keyword, with Solanum lycopersicum (taxon ID: 4081) specified as the organism. Candidate sequences were further screened for the presence of the PSPG (Plant Secondary Product Glycosyltransferase) domain using the NCBI Conserved Domains database. Sequences lacking the PSPG domain were excluded, resulting in a final set of 182 amino acid sequences for phylogenetic analysis. Previously characterized F3GTs and F3GGTs from other species were also included in our phylogenetic analysis. All sequences were aligned using the MUSCLE algorithm. Phylogenetic analysis was performed using the maximum likelihood method with the Goldman +Freq. model and Gamma distribution with 5 categories in MEGA11 software, with 1000 bootstrap replicates. Solyc12g088170 (anthocyanin acyltransferase; S/FdAT1) 85 was used for the outgroup in the phylogenetic analysis.

D. Metabolite analysis using HPLC and LC-MS. Plant samples, including pollen samples from other Solanaceae plants, were collected and immediately frozen in liquid nitrogen. Soluble metabolites in the frozen samples were extracted using 50% methanol at a concentration of 50 mg fresh weight/ml, except for pollen, which was prepared to a concentration of 28.5 mg fresh weight/ml after 15 minutes of sonication at room temperature. The extracts were centrifuged at 10,000 g for 10 minutes to remove plant tissue and insoluble debris before being used for metabolite analysis. Authentic standards used for metabolite identification are kaempferol, kaempferol 3-O-glucoside, kaempferol-3-O-galactoside, kaempferol 3-O-glucoside-6″-O-rhamnoside, quercetin 3-O-glucoside, and quercetin 3-O-galactoside, which were obtained from Chengdu Biopurify Phytochemicals Ltd. (Chengdu, China); quercetin and quercetin 3-O-glucoside-6″-O-rhamnoside from Sigma-Aldrich (St. Louis, MO, USA); and chlorogenic acid from TCI (Portland, OR, USA).

For HPLC analysis, the extracts were analyzed using an UltiMate 3000 HPLC system (Thermo Fisher Scientific, Waltham, MA, USA) equipped with an Acclaim™ RSLC120 C18 column (100 mm×3 mm, 2.2 μm) (Thermo Fisher Scientific, Waltham, MA, USA). The mobile phase consisted of solvent A (0.1% formic acid in water) and solvent B (100% acetonitrile) with the following linear gradient: 5-14% B for 2.2 minutes, 14-18% B for 9 minutes, and 18-95% B for 3.5 minutes, with a flow rate of 0.5 ml/min. Flavonoid contents were quantified based on peak area at 345 nm using authentic standards. K-3-O-gal-2″-O-glu (K2) was quantified equivalently to K-3-O-glu-6″-O-rha.

For Semi-targeted LC-MS analysis, an Ultimate 3000 UHPLC coupled to a TSQ Quantiva triple quadrupole mass spectrometer was used (Thermo Fisher Scientific, Waltham, MA, USA). 3,4,5-trihydroxycinnamic acid from Sigma-Aldrich (St. Louis, MO, USA) was used as an internal standard. The injection volume for all samples was 5 μL. Analytes were eluted on an Agilent InfinityLab Poroshell HPH-C18 column (2.1×150 mm, 1.9 μm particle size) at a column temperature of 40° C. using 0.1% formic acid in water (mobile phase A) and 0.1% formic acid in acetonitrile (mobile phase B). A gradient elution at a flow rate of 0.4 ml/min was applied as follows: 2-42% B for 28 min, 42-100% B for 2 min. The mass spectrometer operated in negative ionization mode. The ESI parameters were as follows: spray voltage (positive), 3500 V; spray voltage (negative), 2500 V; ion transfer tube temperature, 325° C.; vaporizer temperature, 275° C.; sheath gas, 35 Arb; aux gas, 10 Arb; and sweep gas, 0 Arb. Selective reaction monitoring (SRM) was used for metabolite detection. MS/MS parameters were optimized for each metabolite using flow injection analysis of individual standards. Collision-induced dissociation (CID) gas pressure was set at 2 mTorr with a dwell time of 0 msec, and quadrupole resolution (FWHM) was kept at 0.2. Data processing was performed using Xcalibur v4.0 (Thermo Fisher Scientific, Waltham, MA, USA).

To identify the unknown flavonol glycoside (Peak1; K2) lacking an authentic standard, a combination of HPLC and LCMS was employed. K2 was isolated based on its retention time on an HPLC system, and the isolated peak was used for further analysis. Specifically, a Thermo Scientific™ Q Exactive Focus mass spectrometer coupled with a Dionex™ Ultimate™ RSLC 3000 UHPLC system was used for the analysis. The system was equipped with an H-ESI II probe on an Ion Max API Source. Analytes were separated on an Agilent Poroshell 120 EC-C18 column (2.7 μm, 3.0×50 mm) using solvent A (water with 0.1% formic acid) and solvent B (acetonitrile with 0.1% formic acid). The LC program with a flow rate of 0.5 ml/min was as follows: 10% B for 2 min, 10-95% B over 8.5 min, 95% B for 2.5 min, 95% to 10% B in 0.5 min, followed by re-equilibration at 2% B for 2 min. MS signals were acquired under Full MS positive ion mode, covering a mass range of m/z 150-2000, with a resolution of 35,000 and an AGC target of 1E6. The precursor ion (m/z 611.1597) was selected in the Orbitrap, typically with an isolation width of 3.0 m/z and fragmented in the HCD (Higherenergy C-trap dissociation) cell with stepwise a collision energy (CE) of 15.

E. Ubiquinone quantification. Ubiquinone extraction and quantification in tomato samples were modified from Ducluzeau et al86. Tomato pollen samples, 0.8-1.8 mg of pollen grains, were collected and spiked with 3 nmoles of ubiquinone-9. The samples were homogenized in 0.4 ml of 95% (v/v) ethanol using a Pyrex tissue grinder. The grinder was rinsed twice with 0.3 ml of 95% (v/v) ethanol, and washes were combined to the original extract in a 15-ml Pyrex tube containing 0.5 ml of water. The mixture was partitioned twice with 5 ml hexane. Hexane layers were combined, evaporated to dryness with gaseous nitrogen, and resuspended in 0.2 ml of ethanol:dichloromethane (10:1, v/v). Due to the pigment complexity of the pollen extracts, it was not possible to fully resolve the ubiquinol-10 peak. Quinol forms were therefore converted to their respective quinone counterparts by adding 1 μL of 15% H2O2. Samples were then briefly centrifuged (5 min; 18,000 g) and immediately analyzed by HPLC on a 5 μm Supelco Discovery C-18 column (250×4.6 mm, Sigma-Aldrich) thermostated at 30° C. and developed in isocratic mode at a flow rate of 1 ml/min with methanol:hexane (90:10, v/v). Ubiquinone forms were detected spectrophotometrically at 275 nm. Retention times were 13.5 min for ubiquinone-9 and 18 min for ubiquinone-10 (Q10). Data were corrected for recovery of the ubiquinone-9 internal standard and recovery values ranged from 79% to 95%.

F. RNA extraction and expression analysis. Tomato samples were collected and immediately frozen in liquid nitrogen. The samples were then homogenized using a Benchmark BeadBlaster 24 homogenizer (Benchmark Scientific, Edison, NJ, USA). Total RNA was extracted using TRIzol™ reagent (Life Technologies Inc., Gaithersburg, MD, USA).

cDNA synthesis was performed using 2 μg of total RNA and a reverse transcription kit (Thermo Fisher Scientific, Waltham, MA, USA). Quantitative real-time PCR (qRT-PCR) reactions were carried out using a PCR kit (Thermo Fisher Scientific, Waltham, MA, USA). Primer nos. 5 and 6 (AAACAAGGCAATGACACGCC (SEQ ID NO:22) and CCCTGTTTCCTCCTCT-GCTT (SEQ ID NO:23)) were used for S/UGT78D-A, while primer no. 7 and 8 (GACTTCTGCAGCTGGATCAC (SEQ ID NO:24) and AACTCCACCAGGCAAATCAC (SEQ ID NO: 25)) were used for S/UGT78D-B. The expression levels of the target genes were normalized to those of SIACTIN2 (Solyc11g005330) or SIPP2A29 (Solyc05g006590) as internal controls87,88, which were amplified with primer No. 9 and 10 (TTGCTGACCGTATGAGCAAG (SEQ ID NO:26) and GGACAATGGATGGACCAGAC (SEQ ID NO:27)) and primer No. 11 and 12 (CGATGTGTGATCTCCTATGGTC (SEQ ID NO:28) and AAGCTGATGGGCTCTAGA-AATC (SEQ ID NO:29)), respectively.

G. Protein structure prediction. To predict the protein structure of S/UGT78D-B, we employed ColabFold, a computational tool that combines fast homology search with MMseqs2 and the AlphaFold2 algorithm89. The default multiple sequence alignment (MSA) pipeline was used, which included an MMseqs2 search of both the UniRef and environmental sample sequence databases. The resulting predicted protein structure was saved in the Protein Data Bank (PDB) format and visualized using PyMOL 2.5 (Schrödinger, LLC). To determine the ligand position, we overlaid the VvGT1 structure (Worldwide Protein Data Bank), previously resolved via crystallography 7, onto the predicted S/UGT78D-B enzyme structure using PyMOL 2.5.

H. In vitro enzyme assay. The coding sequences (CDS) of wild-type and mutant SIUGT78D genes were synthesized by Twist Biosciences (South San Francisco, CA, USA) and introduced to the pET-28a(+) vector using the NEBuilder HiFi DNA Cloning Kit (NEB, USA). The recombinants were subsequently transformed into E. coli strain BL21 using the heat shock method. For the enzyme assay, the E. coli harboring the expression vector was cultured in 50 ml of Luria broth (LB) medium at 37° C. with shaking at 250 rpm until the optical density at 600 nm (O.D.600) reached approximately 0.5. Isopropyl β-D-1-thiogalactopyranoside (IPTG) was then added to a final concentration of 0.1 mM to induce protein expression. After overnight incubation at 18° C., the cells were harvested by centrifugation at 8000 rpm for 10 minutes at 4° C. The bacterial pellet was resuspended with protein extraction buffer containing 50 mM NaH2PO4·H2O, 300 mM NaCl, 10 mM imidazole, and 0.1% Triton X-100, with the pH adjusted to 8. The resuspended pellet was homogenized using a Benchmark BeadBlaster 24 homogenizer (Benchmark Scientific, Edison, NJ, USA) and centrifuged at 15,000 rpm for 10 minutes to obtain the supernatant. For the enzyme reaction, 100 μL of the crude protein extract was mixed with 100 μL of reaction buffer containing 200 mM Tris-HCl (pH 7.5), 28 mM 2-mercaptoethanol, 200 μM sugar acceptor (flavonol aglycone), and 2 mM UDP-sugar. The total 200 μL reaction mixture was incubated for 30 minutes at 30° C., after which the reaction was stopped by adding 100% methanol. The mixture was then centrifuged for 20 minutes, and 10 μL of the supernatant was injected into HPLC for analysis using the same HPLC running method described in the previous section.

I. Pollen tube growth assay. The pollen germination and pollen tube growth assay were conducted following a previously described protocol with minor modifications. Tomato pollen grains were collected from newly opened flowers in the morning and immediately used the same day to preserve viability. The collected pollen was incubated in Pollen Germination Medium (PGM) consisting of 24% (w/v) PEG4000, 0.01% (w/v) boric acid, 2% (w/v) sucrose, 20 mM HEPES buffer (pH 6.0), 30 mM Ca (NO3)2, 1.5 mM MgSO4, and 1 mM KNO3, on slide glasses topped with cover slips. After 4 hours of incubation at 22° C. in the dark, the images of pollen tubes were captured using an Olympus IX81-DSU Spinning Disk Confocal Microscope (Olympus America Inc., Center Valley, PA, USA). To minimize potential discrepancies in incubation times, at least three replicate slides for each condition and for genotype were prepared and the order of capturing confocal microscope images was rotated in each replicate and each batch. At least three independent batches were examined. Pollen tube length and the number of ruptured or intact pollen tubes were measured using ImageJ (ver. 1.54g for MacOS). For the flavonol feeding assay, a stock solution of flavonols dissolved in DMSO was added to PGM to achieve the desired final concentration, while a DMSO-only solution was used as a mock control.

1. A genetically modified plant comprising a modified UGT78D-B gene.

2. The genetically modified plant of claim 1, wherein the modified UGT78D-B gene comprises a deletion, insertion, substitution, or frameshift mutation of the UGT78D-B gene.

3. The genetically modified plant of claim 2, wherein the genetically modified plant has:
(a) decreased expression of the UGT78D-B gene; and/or
(b) decreased activity of a protein encoded by the UGT78D-B gene.

4. The genetically modified plant of claim 3, wherein the genetically modified plant has decreased male fertility.

5. The genetically modified plant of claim 4, wherein pollen tube growth from pollen produced from the plant is reduced by at least 25%, at least 30%, at least 35%, at least 40%, at least 45%, at least 50%, at least 60%, at least 65%, at least 70%, at least 75%, at least 80%, at least 85%, at least 90%, or at least 95% relative to pollen tube grown from pollen produced by a similar plant that does not contain a modified UGT78D-B gene.

6. The genetically modified plant of claim 4, wherein the genetically modified plant is a Solanaceae plant, optionally wherein the Solanaceae plant is a tomato plant, a tobacco plant, a pepper plant, or a potato plant.

7. A CRISPR system for decreasing male fertility in a plant comprising an RNA-guided DNA endonuclease or a nucleic acid encoding the RNA-guided DNA endonuclease and a guide RNA (gRNA) or a nucleic acid encoding the gRNA into the plant, wherein the RNA-guided DNA endonuclease and the gRNA form a complex that targets the genomic UGT78D-B locus.

8. The CRISPR system of claim 7, wherein:
(a) the CRISPR system comprises a CRISPR class 1 system, a CRISPR class 2 system, a CRISPR/Cas system, a CRISPR/Cas9 system, a CRISPR/zCas9 system or a CRISPR/Cas3 system; and
(b) the gRNA comprises a sequence that hybridizes with a sequence present in a genomic UGT78D-B locus.

9. The CRISPR system of claim 7, wherein the gRNA comprises a sequence that hybridizes to a sequence present in any of SEQ ID NOs: 1, 2, 3, 4, and 8, or a complement thereof, or an ortholog thereof.

10. The CRISPR system of claim 9, wherein the gRNA comprises any of SEQ ID NOs: 10-16 or an RNA equivalent thereof.

11. A method of modifying male fertility in a plant, the method comprising modifying activity of a UGT78D-B protein in the plant.

12. The method of claim 11, wherein modifying activity of a UGT78D-B comprises:
(a) reducing expression of an endogenous UGT78D-B gene in the plant; or
(b) reducing activity of UGT78D-B in the plant.

13. The method of claim 12, wherein reducing expression of the UGT78D-B gene or reducing activity of UGT78D-B comprises:
(a) introducing an RNA interference (RNAi) molecule or an antisense nucleotide targeting an UGT78D-B mRNA into the plant or expressing an RNAi nucleic acid sequence or an antisense nucleic acid sequence targeting the UGT78D-B mRNA in the plant; or
(b) genetically modifying the plant to form a deletion, insertion, substitution, or frameshift mutation in an endogenous UGT78D-B gene,
(c) genetically modifying an endogenous UGT78D-B gene in the plant, wherein the genetic modification results in loss of enzymatic activity of the UGT78D-B encoded by endogenous UGT78D-B gene.

14. The method of claim 13, wherein genetically modifying the endogenous UGT78D-B gene comprises introducing a CRISPR system into a plant cell, wherein the CRISPR system comprises an RNA-guided DNA endonuclease or a nucleic acid encoding the RNA-guided DNA endonuclease and a gRNA or a nucleic acid encoding the gRNA into the plant, wherein the RNA-guided DNA endonuclease and the gRNA form a complex that targets the UGT78D-B genomic locus.

15. The method of claim 14, wherein the gRNA comprises a sequence that hybridizes with a sequence present in any of SEQ ID NOs: 1, 2, 4, 6, and 8, or a complement thereof, or an ortholog thereof.

16. The method of claim 15, wherein:
(a) the plant is a tomato plant and the gRNA comprises SEQ ID NO: 10 or an RNA equivalent thereof;
(b) the plant is a tobacco plant and the gRNA comprises SEQ ID NO:11 or 12, or an RNA equivalent thereof;
(c) the plant is a pepper plant and the gRNA comprises SEQ ID NO: 13 or 14, or an RNA equivalent thereof; or
(d) the plant is a potato plant and the gRNA comprises SEQ ID NO:15 or 16, or an RNA equivalent thereof.

17. The method of claim 12, wherein the plant has reduced pollen tube growth compared to a similar plant that has not been modified to reduce expression of an endogenous UGT78D-B gene or reduced activity of UGT78D-B.

18. The method of claim 17, wherein the pollen tube growth is reduced by at least 25%, at least 30%, at least 35%, at least 40%, at least 45%, at least 50%, at least 60%, at least 65%, at least 70%, at least 75%, at least 80%, at least 85%, at least 90%, or at least 95%.

19. The method of claim 14, wherein the method further comprises generating one or more regenerants from the plant cell.

20. The method of claim 11, wherein the plant is a Solanaceae plant, a tomato plant, a tobacco plant, a pepper plant, or a potato plant.
